(19) United States
(12) Patent Application Publication
(10) Pub. No.: US 2026/0137629 A1
(43) Pub. Date: May 21, 2026

(54) Novel Lipids For Delivery Of Nucleic Acid Segments

(71) Applicant: ASTRAZENECA AB, Sodertalje (SE)

(72) Inventors: David ULKOSKI, Wilmington, DE (US); Lennart LINDFORS, Sodertalje (SE); Venkata R. KRISHNAMURTHY, Wilmington, DE (US)

(21) Appl. No.: 18/704,133

(22) PCT Filed: Oct. 24, 2022
(86) PCT No.: PCT/IB2022/060200

§ 371 Date: Apr. 24, 2024

Related U.S. Application Data
(60) Provisional application No. 63/271,960, filed on Oct. 26, 2021.

(51) Int. Cl. A61K 9/51; A61K 9/00; A61K 31/7105; A61K 47/22; C07D 498/08

(52) U.S. Cl. CPC A61K 9/5123; A61K 9/0019; A61K 9/0078; A61K 9/5192; A61K 31/7105; A61K 47/22; C07D 498/08

(57) ABSTRACT

Disclosed herein are compounds of Formula (I) or pharmaceutically acceptable salts thereof, wherein X¹, Y¹, X², Y², a, b, c, d, e and f are as defined herein. Also disclosed are lipid nanoparticles comprising a compound of Formula (I) or a pharmaceutically acceptable salt thereof; pharmaceutical compositions comprising a plurality of lipid nanoparticles comprising a compound of Formula (I) or a pharmaceutically acceptable salt thereof and a nucleic acid segment; as well as methods for delivering a nucleic acid segment comprising administering a plurality of lipid nanoparticles comprising a compound of Formula (I) or a pharmaceutically acceptable salt thereof, and a nucleic acid segment.

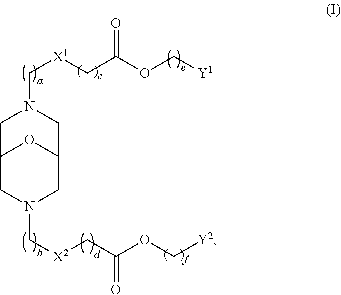

### CROSS-REFERENCE TO RELATED APPLICATION

This application claims the benefit of priority to U.S. Provisional Application No. 63/271,960, filed on Oct. 26, 2021, which is incorporated by reference herein in its entirety for all purposes.

### BACKGROUND

Nucleic acid segments such as oligonucleotides (e.g., RNA, for example, messenger RNAs [mRNAs] and small interfering RNAs [siRNAs], antisense oligonucleotides [ASOs] and DNA) have broad potential as new therapeutic treatments for a variety of diseases and disorders. However, challenges remain in administering oligonucleotide therapeutics. Typical formulations include encapsulating the oligonucleotide into a lipid nanoparticle (LNP). LNP formulations usually include (a) an ionizable or cationic lipid or polymeric material bearing a tertiary or quaternary amine to encapsulate the polyanionic mRNA; (b) a zwitterionic lipid that resembles the lipids in the cell membrane; (c) cholesterol to stabilize the lipid bilayer of the LNP; and (d) a polyethylene glycol (PEG)-lipid to give the nanoparticle a hydrating layer, improve colloidal stability and reduce protein absorption. (see Kowalski et al., Molecular Therapy, 27(4), (2019), 710-728).

In 2018, the FDA approved the first RNA interference therapy, patisiran, for the treatment of polyneuropathy in people with hereditary transthyretin-mediated amyloidosis, which is intravenously delivered using an LNP that incorporates an ionizable lipid (DLin-MC3-DMA, [MC3]). MC3, however, might not be most suitable for all delivery systems, depending on the targeted organ, intended delivery route and required therapeutic window. Dose-limiting toxicities were reported from studies in two toxicology-relevant test species, rat and monkey, that were related to MC3-based LNP formulation rather than the delivered cargo. (see Sedic et al., Vet. Pathol. 55(2), (2018), 341-354). Recently, lipid nanoparticle technology has also successfully been applied to generate the first approved mRNA products for prophylactic vaccination against SARS-COV-2 virus (see e.g. L. Schoenmaker et al., International Journal of Pharmaceutics, 601, (2021), May 120586). However, there remains a need to develop new ionizable lipids for use in lipid nanoparticle formulations for delivery of oligonucleotide therapeutics.

### SUMMARY

In some embodiments, disclosed is a compound of Formula (I):

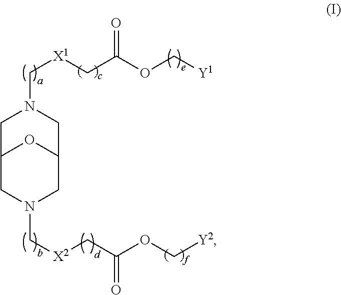

or a pharmaceutically acceptable salt thereof, wherein
a and b are each independently 3, 4 or 5;
c and d are each independently 1, 2 or 3;
e and f are each independently 0, 1 or 2;
X¹ is methylene or

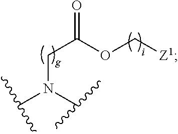

X² is methylene or

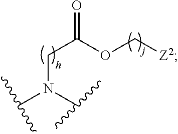

g and h are each independently 1, 2 or 3;
i and j are each independently 0, 1 or 2;
Y¹ and Y² are each independently straight-chain C₇₋₁₀ alkyl, straight-chain C₇₋₁₀ alkenyl, or

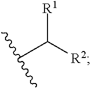

Z¹ and Z² are each independently straight-chain C₇₋₁₀ alkyl, straight-chain C₇₋₁₀ alkenyl, or

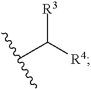

R¹ and R² are each independently straight-chain C₇₋₁₀ alkyl; and
R³ and R⁴ are each independently straight-chain C₇₋₁₀ alkyl.

In some embodiments, disclosed is a lipid nanoparticle comprising a compound of Formula (I), or a pharmaceutically acceptable salt thereof.

In some embodiments, disclosed is a pharmaceutical composition comprising a plurality of lipid nanoparticles comprising a compound of Formula (I) or a pharmaceutically acceptable salt thereof and a nucleic acid segment.

In some embodiments, disclosed is a method of treating a disease or disorder in a subject, comprising administering to the subject a therapeutically effective amount of a pharmaceutical composition as described herein.

In some embodiments, disclosed is a pharmaceutical composition as described herein for use in the treatment of a disease or disorder.

### BRIEF DESCRIPTION OF THE DRAWINGS

FIG. 1 illustrates the expression of eGFP in rats lung at 24 hours after intratracheal administration of LNP formulations comprising MC3, MOD5, Compound 4 and Compound 2.

FIG. 2 illustrates the BALF neutrophil concentration in rat BALF at 24 hours after intratracheal administration of LNP formulations comprising MC3, MOD5, Compound 4 and Compound 2.

FIG. 3 illustrates the expression of eGFP in rats lung at 24 hours after intratracheal administration of LNP formulations comprising MC3 and Compound 1.

FIG. 4 illustrates the BALF neutrophil concentration in rat BALF at 24 hours after intratracheal administration of LNP formulations comprising MC3 and Compound 1.

FIGS. 5A and 5B illustrate the expression of eGFP as shown by IHC in both macrophages and type 1 epithelial cells after intratracheal administration of LNP formulations comprising Compound 1.

FIG. 6 illustrates the level of eGFP in rat lung at 5 and 24 hours after inhalation administration of LNP formulation comprising Compound 1.

FIG. 7 illustrates the BALF neutrophil concentration in rat BALF at 24 hours after inhalation administration of LNP formulations comprising Compound 1.

FIG. 8 illustrates the expression of eGFP in mouse liver at 24 hours after intravenous administration of LNP formulations comprising MC3, Compound 2 and Compound 3.

FIG. 9 illustrates the expression of eGFP in mouse spleen at 24 hours after intravenous administration of LNP formulations comprising MC3, Compound 2 and Compound 3.

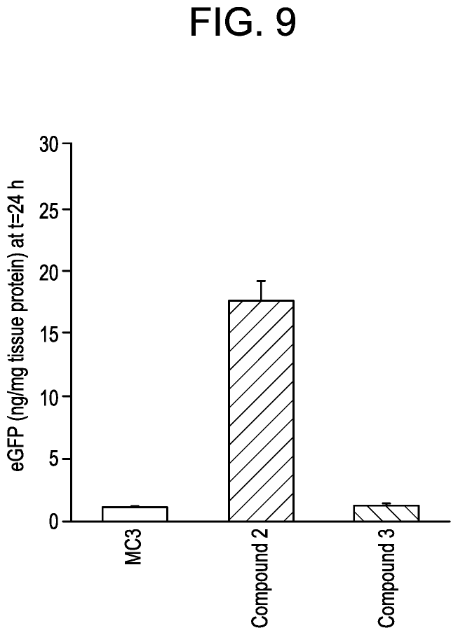

FIG. 10 illustrates the expression of eGFP in mouse lung at 24 hours after intravenous administration of LNP formulations comprising MC3, Compound 2 and Compound 3.

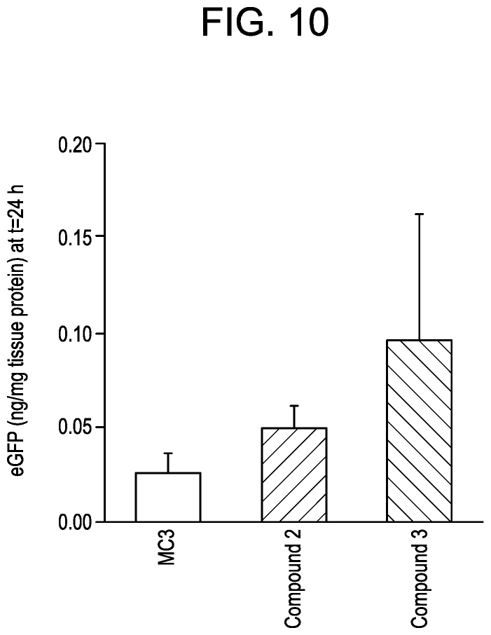

FIG. 11 illustrates the expression of eGFP in mouse kidney at 24 hours after intravenous administration of LNP formulations comprising MC3, Compound 2 and Compound 3.

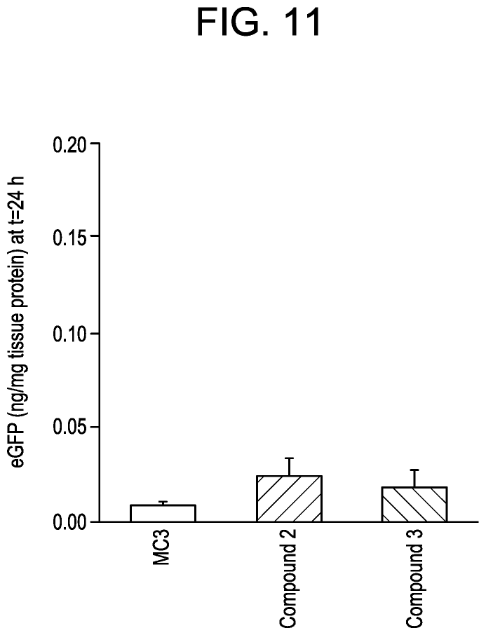

FIG. 12 illustrates the expression of eGFP in mouse heart at 24 hours after intravenous administration of LNP formulations comprising MC3, Compound 2 and Compound 3.

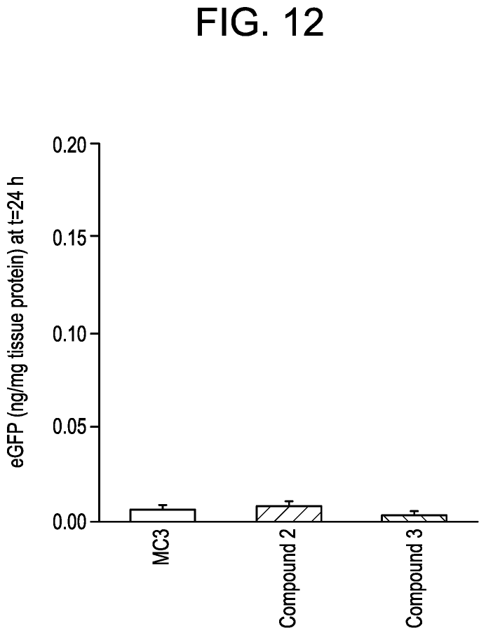

FIG. 13 illustrates the expression of luciferase protein in mouse liver at 24 hours after intravenous administration of LNP formulations comprising MC3, Compound 2 and Compound 3.

FIG. 14 illustrates the IVIS images of mice heart, lung, spleen and liver at 24 hours after intravenous administration of LNP formulations comprising MC3, Compound 2, Compound 3.

FIG. 15 illustrates the hEPO protein concentration in mouse plasma at 6 hours after intravenous administration of LNP formulations comprising MC3, Compound 2 and Compound 3.

FIG. 16 illustrates the cortex and striatum average Luc expression in LoxP Luc reporter mouse after intrastriatal administration of LNP formulations comprising MOD5 and Compound 5.

### DETAILED DESCRIPTION

In some embodiments, disclosed is a compound of Formula (I):

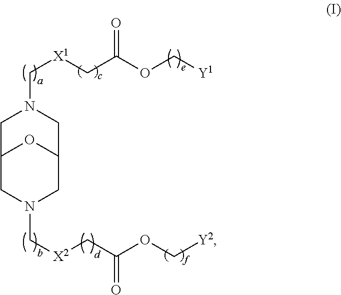

or a pharmaceutically acceptable salt thereof, wherein
a and b are each independently 3, 4 or 5;
c and d are each independently 1, 2 or 3;
e and f are each independently 0, 1 or 2;
X¹ is methylene or

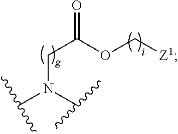

X² is methylene or

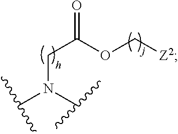

g and h are each independently 1, 2 or 3;
i and j are each independently 0, 1 or 2;
Y¹ and Y² are each independently straight-chain C₇₋₁₀ alkyl, straight-chain C₇₋₁₀ alkenyl, or

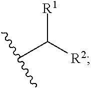

Z¹ and Z² are each independently straight-chain C₇₋₁₀ alkyl, straight-chain C₇₋₁₀ alkenyl, or

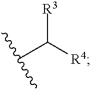

R¹ and R² are each independently straight-chain C₇₋₁₀ alkyl; and
R³ and R⁴ are each independently straight-chain C₇₋₁₀ alkyl.

In some embodiments of Formula (I), X¹ and X² are both methylene.

In some embodiments of Formula (I), a and b are both 4; and c and d are both 2.

In some embodiments of Formula (I), e and f are both 0.

In some embodiments of Formula (I),
Y¹ is straight-chain C₇₋₁₀ alkyl or straight-chain C₇₋₁₀ alkenyl;
Y² is

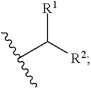

and
R¹ and R² are each independently straight-chain C₇₋₁₀ alkyl.

In some embodiments of Formula (I), Y¹ and Y² are each independently

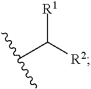

and
R¹ and R² are each independently straight-chain C₇₋₁₀ alkyl.

In some embodiments of Formula (I), e and f are each independently 1 or 2. In some further embodiments of Formula (I), Y¹ and Y² are each independently

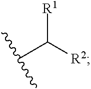

and
R¹ and R² are each independently straight-chain C₇₋₁₀ alkyl.

In some embodiments, the compound of Formula (I) is a compound of Formula (11):

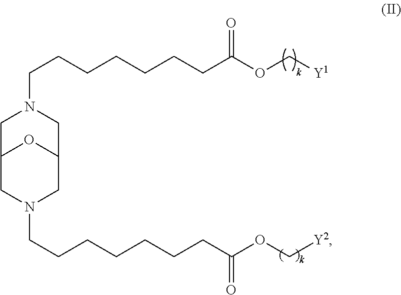

wherein
k is 0, 1 or 2;
Y¹ is straight-chain C₇₋₁₀ alkyl, straight-chain C₇₋₁₀ alkenyl, or

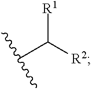

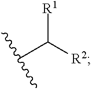

R¹ and R² are each independently straight-chain C₇₋₁₀ alkyl.

In some embodiments of Formula (I),
X¹ is

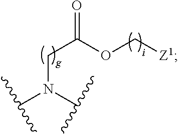

and
X² is

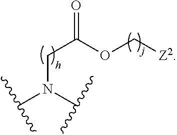

In some embodiments of Formula (I), a and b are both 4; and c and d are both 2.

In some embodiments of Formula (I), e and f are each independently 1 or 2.

In some embodiments of Formula (I), g and h are both 2.

In some embodiments of Formula (I), i and j are both 1.

In some embodiments of Formula (I), Y¹ and Y² are each independently

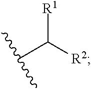

and
R¹ and R² are each independently straight-chain C₇₋₁₀ alkyl.

In some embodiments, the compound of Formula (I) is heptadecan-9-yl 8-(7-(8-(nonyloxy)-8-oxooctyl)-9-oxa-3,7-diazabicyclo[3.3.1]nonan-3-yl)octanoate, or a pharmaceutically acceptable salt thereof.

In some embodiments, the compound of Formula (I) is heptadecan-9-yl (Z)-8-(7-(8-(non-2-en-1-yloxy)-8-oxooctyl)-9-oxa-3,7-diazabicyclo[3.3.1]nonan-3-yl)octanoate, or a pharmaceutically acceptable salt thereof.

In some embodiments, the compound of Formula (I) is 3-heptyldodecyl 8-(7-(8-((3-octylundecyl)oxy)-8-oxooctyl)-9-oxa-3,7-diazabicyclo[3.3.1]nonan-3-yl)octanoate, or a pharmaceutically acceptable salt thereof.

In some embodiments, the compound of Formula (I) is di(heptadecan-9-yl) 8,8′-(9-oxa-3,7-diazabicyclo[3.3.1]nonane-3,7-diyl)dioctanoate, or a pharmaceutically acceptable salt thereof.

In some embodiments, the compound of Formula (I) is tetrakis(2-octyldecyl) 3,3′,3,3′-(((9-oxa-3,7-diazabicyclo[3.3.1]nonane-3,7-diyl)bis(butane-4,1-diyl))bis(azanetriyl))tetrapropionate, or a pharmaceutically acceptable salt thereof.

In some embodiments, the compound of Formula (I) is heptadecan-9-yl 8-(7-(8-(nonyloxy)-8-oxooctyl)-9-oxa-3,7-diazabicyclo[3.3.1]nonan-3-yl)octanoate.

In some embodiments, the compound of Formula (I) is heptadecan-9-yl (Z)-8-(7-(8-(non-2-en-1-yloxy)-8-oxooctyl)-9-oxa-3,7-diazabicyclo[3.3.1]nonan-3-yl)octanoate.

In some embodiments, the compound of Formula (I) is 3-heptyldodecyl 8-(7-(8-((3-octylundecyl)oxy)-8-oxooctyl)-9-oxa-3,7-diazabicyclo[3.3.1]nonan-3-yl)octanoate.

In some embodiments, the compound of Formula (I) is di(heptadecan-9-yl) 8,8′-(9-oxa-3,7-diazabicyclo[3.3.1]nonane-3,7-diyl)dioctanoate.

In some embodiments, the compound of Formula (I) is tetrakis(2-octyldecyl) 3,3′,3,3′-(((9-oxa-3,7-diazabicyclo[3.3.1]nonane-3,7-diyl)bis(butane-4,1-diyl))bis(azanetriyl))tetrapropionate.

As used herein, the term “Ci-j” indicates a range of the carbon atoms numbers, wherein i and j are integers and the range of the carbon atoms numbers includes the endpoints (i.e. i and j) and each integer point in between, and wherein j is greater than i. For examples, C₇₋₁₀ indicates a range of seven to ten carbon atoms, including seven carbon atom, eight carbon atoms, nine carbon atoms and ten carbon atoms.

As used herein, the term “alkyl”, whether as part of another term or used independently, refers to a saturated hydrocarbon chain. In one embodiment, the saturated hydrocarbon chain mentioned above is straight-chain alkyl in which the carbon atoms are connected in one continuous chain with no branches.

The term “Ci-j alkyl” refers to an alkyl having i to j carbon atoms. For example, C₇₋₁₀ alkyl refers to an alkyl having 7 to 10 carbon atoms.

As used herein, the term “alkenyl”, whether as part of another term or used independently, refers to an unsaturated hydrocarbon chain containing at least one double bond. In one embodiment, the unsaturated hydrocarbon chain mentioned above is straight-chain alkenyl in which the carbon atoms are connected in one continuous chain with no branches. The term “Ci-j alkenyl” refers to an alkenyl having i to j carbon atoms. For example, C₇₋₁₀ alkenyl refers to an alkenyl having 7 to 10 carbon atoms. In one embodiment, the C₇₋₁₀ alkenyl group contains one double bond.

In some embodiments, disclosed is a compound of Formula (I). In some embodiments, disclosed is a pharmaceutically acceptable salt of the compound of Formula (I). The term “pharmaceutically acceptable salt” includes acid addition salts that retain the biological effectiveness and properties of the compound of Formula (I) and, which typically are not biologically or otherwise undesirable.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulfate, naphthoate, napsylate, nicotinate, nitrate, octadecanoate, oleate, oxalate, palmitate, palmoate, phosphate/hydrogen phosphate/dihydrogen phosphate, polygalacturonate, propionate, stearate, succinate, subsalicylate, sulfate/hydrogensulfate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic acid, sulfosalicylic acid, and the like.

In some embodiments, disclosed are lipid nanoparticles (LNPs) comprising the compound of Formula (I), or a pharmaceutically acceptable salt thereof. In some embodiments, disclosed are lipid nanoparticles (LNPs) comprising the compound of Formula (I). The term “lipid nanoparticle” includes an electron dense nanostructural core produced by microfluidic mixing of lipid-containing solutions in ethanol with aqueous solutions. The lipid nanoparticles disclosed herein may be constructed from any materials used in conventional nanoparticle technology, for example, ionizable lipids, neutral lipids, sterols and polymer-conjugated lipids, provided that the net charge of the nanoparticle is about zero.

In some embodiments, the compound of Formula (I) is the ionizable lipid suitable for lipid nanoparticles. Other non-limiting examples of ionizable lipids that may be combined with the compound of Formula (I) in a lipid nanoparticle include, for instance, lipids containing a positive charge at the acidic scale of physiological pH range, for example 1,2-dilinoleyloxy-3-dimethylaminopropane (DLin-DMA), dilinoleylmethyl-4-dimethylaminobutyrate (DLin-MC3-DMA, (see e.g., U.S. Pat. No. 8,158,601), 2-dilinoleyl-4-dimethylaminoethyl-[1,3]-dioxolane (DLin-KC2-DMA), Merck-32 (see e.g., WO 2012/018754), Acuitas-5 (see e.g., WO 2015/199952), KL-10 (see e.g., U.S. Patent Application Publication 2012/0295832), C12-200 (see e.g., Love, K T et al., PNAS, 107: 1864 (2009)), and the like. The ionizable lipids may be present in an amount ranging from about 5% to about 90%, such as from about 10% to about 80%, for instance from about 25% to about 75%, for example, from about 40% to about 60%, from about 40% to about 50%, such as about 45% or about 50%, molar percent, relative to the total lipid present in the lipid nanoparticles.

The term “neutral lipid” includes lipids that have a zero-net charge at physiological pH, for example, lipids that exist in an uncharged form or neutral zwitterionic form at physiological pH, such as distearoyl phosphatidylcholine (DSPC), dioleoyl phosphatidylethanolamine (DOPE), dipalmitoyl phosphatidylcholine (DPPC), dimyristoyl phosphatidylcholine (DMPC), and the like, and combinations thereof. The neutral lipids may be present in an amount ranging from about 1% to about 50%, such as from about 5% to about 20%, for example, 7.5% to about 12.5%, for instance, about 10%, molar percent, relative to the total lipid present in the lipid nanoparticles. In some embodiments, the neutral lipid is DSPC. In some embodiments, the neutral lipid is DOPE. In some embodiments, the neutral lipid is DPPC. In some embodiments, the neutral lipid is DMPC.

The term “sterol” includes cholesterol, and the like. The sterols may be present in an amount ranging from about 10% to about 90%, such as from about 20% to about 50%, for instance, from about 35%-45%, such as about 38.5%, molar percent, relative to the total lipid present in the lipid nanoparticles. In some embodiments, the sterol is cholesterol.

The term “polymer-conjugated lipid” includes lipids that comprise a lipid portion and a polymer portion, such as pegylated lipids comprising both a lipid portion and a polyethylene glycol portion. Non-limiting examples include dimyristoyl phosphatidyl ethanolamine-poly(ethylene glycol) 2000 (DMPE-PEG2000), DPPE-PEG2000, DMG-PEG2000, DPG-PEG2000, PEG2000-c-DOMG, PEG2000-c-DOPG, and the like. The molecular weight of the poly(ethylene glycol) that may be used may range from about 500 and about 10,000 Da, or from about 1,000 to about 5,000 Da. In some embodiments, the polymer-conjugated lipid is DMPE-PEG2000. In some embodiments, the polymer-conjugated lipid is DPPE-PEG2000. In some embodiments, the polymer-conjugated lipid is DMG-PEG2000. In some embodiments, the polymer-conjugated lipid is DPG-PEG2000. In some embodiments, the polymer-conjugated lipid is PEG2000-c-DOMG. In some embodiments, the polymer-conjugated lipid is PEG2000-c-DOPG. The polymer-conjugated lipids may be present in an amount ranging from about 0% to about 20%, for example about 0.5% to about 5%, such as about 1% to about 2%, for instance, about 1.5%, molar percent, relative to the total lipid present in the lipid nanoparticles.

In at least one embodiment of the present disclosure, lipid nanoparticles may be prepared by combining multiple lipid components. For example, the lipid nanoparticles may be prepared combining the compound of Formula (I), or a pharmaceutically acceptable salt thereof, a sterol, a neutral lipid, and a polymer-conjugated lipid at a molar ratio of 50:40-x:10:x, with respect to the total lipids present. For example, the lipid nanoparticles may be prepared combining the compound of Formula (I), or a pharmaceutically acceptable salt thereof, a sterol, a neutral lipid, and a polymer-conjugated lipid at a molar ratio of 50:37:10:3 (mol/mol), or, for instance, a molar ratio of 50:38.5:10:1.5 (mol/mol), or, for example, 50:39.5:10:0.5 (mol/mol), or 50:39.75:10:0.25 (mol/mol).

In another embodiment, a lipid nanoparticle may be prepared using the compound of Formula (I), or a pharmaceutically acceptable salt thereof, a sterol (such as cholesterol), a neutral lipid (such as DSPC), and a polymer conjugated lipid (such as DMPE-PEG2000) at a molar ratio of about 50:38.5:10:1.5 (mol/mol), with respect to the total lipids present. Yet another non limiting example is a lipid nanoparticle comprising the compound of Formula (I), or a pharmaceutically acceptable salt thereof, a sterol (such as cholesterol), a neutral lipid (such as DSPC), and a polymer conjugated lipid (such as DMPE-PEG2000) at a molar ratio of about 47.7:36.8:12.5:3 (mol/mol), with respect to the total lipids present. Another non-limiting example is a lipid nanoparticle comprising the compound of Formula (I), or a pharmaceutically acceptable salt thereof, a sterol (such as cholesterol), a neutral lipid (such as DSPC), and a polymer conjugated lipid (such as DMPE-PEG2000) at a molar ratio of about 52.4:40.4:6.4:0.8 (mol/mol), with respect to the total lipids present. In another embodiment, a non-limiting example is a lipid nanoparticle comprising the compound of Formula (I), or a pharmaceutically acceptable salt thereof, a sterol (such as cholesterol), a neutral lipid (such as DSPC), and a polymer conjugated lipid (such as DMPE-PEG2000) at a molar ratio of about 53.5:41.2:4.6:0.7 (mol/mol), with respect to the total lipids present. Another non-limiting example is a lipid nanoparticle comprising the compound of Formula (I), or a pharmaceutically acceptable salt thereof, a sterol (such as cholesterol), a neutral lipid (such as DSPC), and a polymer conjugated lipid (such as DMPE-PEG2000) at a molar ratio of about 30:50:19:1 (mol/mol), with respect to the total lipids present.

The selection of neutral lipids, sterols, and/or polymer-conjugated lipids that comprise the lipid nanoparticles, as well as the relative molar ratio of such lipids to each other, may be determined by the characteristics of the selected lipid(s), the nature of the intended target cells, and the characteristics of the nucleic acid segment to be delivered. For instance, in certain embodiments, the molar percent of the compound of Formula (I), or a pharmaceutically acceptable salt thereof, in the lipid nanoparticle may be greater than about 10%, greater than about 20%, greater than about 30%, greater than about 40%, greater than about 50%, greater than about 60%, or greater than about 70%, relative to the total lipids present. The molar percent of neutral lipid in the lipid nanoparticle may be greater than about 5%, greater than about 10%, greater than about 20%, greater than about 30%, or greater than about 40%, relative to the total lipids present. The molar percent of sterol in the lipid nanoparticle may be greater than about 10%, greater than about 20%, greater than about 30%, or greater than about 40%, relative to the total lipids present. The molar percent of polymer-conjugated lipid in the lipid nanoparticle may be greater than about 0.25%, such as greater than about 1%, greater than about 1.5%, greater than about 2%, greater than about 5%, or greater than about 10%, relative to the total lipids present.

According to the present disclosure, the lipid nanoparticles may comprise each of the compound of Formula (I), or a pharmaceutically acceptable salt thereof, neutral lipids, sterols, and/or polymer-conjugated lipids in any useful orientation desired. For example, the core of the nanoparticle may comprise the compound of Formula (I), or a pharmaceutically acceptable salt thereof, alone or in combination with another ionizable lipid, a sterol and one or more layers comprising neutral lipids and/or polymer-conjugated lipids may subsequently surround the core. For instance, according to one embodiment, the core of the lipid nanoparticle may comprise a core comprising the compound of Formula (I), or a pharmaceutically acceptable salt thereof, and a sterol (e.g., cholesterol) in any particular ratio, surrounded by a neutral lipid monolayer (e.g., DSPC) of any particular thickness, further surrounded by an outer polymer-conjugated lipid monolayer of any particular thickness. In such examples, the nucleic acid segment may be incorporated into any one of the core or subsequent layers depending upon the nature of the intended target cells, and the characteristics of the nucleic acid segment to be delivered.

The core and outer layers may further comprise other components typically incorporated into lipid nanoparticles known in the art. Furthermore, it is understood by one skilled in the art that liposomes are delivery vehicles that possess a vesicular structure distinct from the lipid nanoparticles as disclosed herein. The liposome vesicles are composed of a lipid bilayer that forms in the shape of a hollow sphere encompassing an aqueous phase. For example, liposomes contain the lamellar phase while the lipid nanoparticles have non-lamellar structures.

In addition, the molar percent of the components of the lipid nanoparticle (e.g., the compound of Formula (I), or a pharmaceutically acceptable salt thereof, neutral lipids, sterols, and/or polymer-conjugated lipids) that comprise the lipid nanoparticles may be selected in order to provide a particular physical parameter of the overall lipid nanoparticle, such as the surface area of one or more of the lipids. For example, the molar percent of the compound of Formula (I), or a pharmaceutically acceptable salt thereof, neutral lipids, sterols, and/or polymer-conjugated lipids that comprise the lipid nanoparticles may be selected to yield a surface area per neutral lipid, for example, DSPC. By way of non-limiting example, the molar percent of the compound of Formula (I), or a pharmaceutically acceptable salt thereof, neutral lipids, sterols, and/or polymer-conjugated lipids may be determined to yield a surface area per DSPC of about 1.0 nm² to about 2.0 nm², for example about 1.2 nm².

According to the present disclosure, the lipid nanoparticles may further comprise a nucleic acid segment, which may be associated on the surface of the lipid nanoparticles and/or encapsulated within the same lipid nanoparticles.

The term “nucleic acid segment” is understood to mean any one or more nucleic acid segments selected from antisense oligonucleotides, DNA, mRNAs, siRNAs, Cas9guided-RNA complex, or combinations thereof. The nucleic acid segments herein may be wildtype or modified. In at least one embodiment, the lipid nanoparticles may comprise a plurality of different nucleic acid segments. In yet another embodiment, the nucleic acid segment, wildtype or modified, encodes a polypeptide of interest.

A modified nucleic acid segment includes nucleic acid segments with chemical modifications to any part of the structure such that the nucleic acid segment is not naturally occurring. In some embodiments, the nucleic acid segment is an RNA. In some embodiments, the nucleic acid segment is an mRNA. In some embodiments, the nucleic acid segment is a modified mRNA.

The term “therapeutically effective amount” as used herein refers to an amount of nucleic acid segment sufficient to modulate protein expression in a target tissue and/or cell type. In some embodiments, a therapeutically effective amount of the nucleic acid segment is an amount sufficient to treat a disease or disorder associated with the protein expressed by the nucleic acid segment.

In at least one embodiment, the weight ratio of total lipid phase to nucleic acid segment ranges from about 40:1 to about 1:1, such as about 10:1. This corresponds to an approximate molar ratio of the compound of Formula (I), or a pharmaceutically acceptable salt thereof, to nucleic acid monomer of about 3:1. In yet another example, the weight ratio of total lipid phase to nucleic acid segment ranges from about 30:1 to about 1:1, such as about 20:1, which corresponds to an approximate molar ratio of the compound of Formula (I), or a pharmaceutically acceptable salt thereof, to nucleic acid monomer of about 6:1. However, the relative molar ratio of lipid phase and/or lipid phase components to the nucleic acid monomer may be determined by the nature of the intended target cells and characteristics of nucleic acid segment and thus, are not limited in scope to the above-identified embodiments. In some embodiments, the molar ratio of the compound of Formula (I), or a pharmaceutically acceptable salt thereof, to nucleic acid monomer is from about 2.75:1 to 6:1. In some embodiments, the molar ratio of the compound of Formula (I), or a pharmaceutically acceptable salt thereof, to nucleic acid monomer is about 2.75:1. In some embodiments, the approximate molar ratio of the compound of Formula (I), or a pharmaceutically acceptable salt thereof, to nucleic acid monomer of about 3:1. In some embodiments, the molar ratio of the compound of Formula (I), or a pharmaceutically acceptable salt thereof, to nucleic acid monomer is about 5.5:1. In some embodiments, the approximate molar ratio of the compound of Formula (I), or a pharmaceutically acceptable salt thereof, to nucleic acid monomer of about 6:1.

In some embodiments, the lipid nanoparticles have a z-average particle diameter (<d>z) of about 200 nm or less, for example, less than or equal to about 100 nm, or, for instance, less than or equal to about 75 nm. In at least one embodiment of the present disclosure, the lipid nanoparticles have a z-average particle diameter ranging from about 50 nm to about 100 nm, for example, about 60 nm to about 90 nm, from about 70 nm to about 80, such as about 75 nm.

In certain embodiments, the lipid nanoparticles have an encapsulation efficiency (% EE) of nucleic acid segments of about 80% or higher, such as higher than about 90%, such as ranging from about 95%-100%. As used herein, the term “encapsulation efficiency” refers to the ratio of encapsulated nucleic acid segment in the lipid nanoparticles to total nucleic acid segment content in the lipid nanoparticle composition measured by lysis of the lipid nanoparticles using a detergent, e.g., Triton X-100.

Pharmaceutical compositions of the present disclosure may further comprise at least one pharmaceutically acceptable carrier. As used herein, the term “pharmaceutically acceptable carrier” includes compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutical compositions may be in a form suitable for parenteral administration. For example, a suitable parenteral administration include, but is not limited to, subcutaneous administration, intramuscular administration, and intravenous administration. The pharmaceutical compositions may be in a form suitable for intratracheal instillation, bronchial instillation, and/or inhalation. Pharmaceutical liquid compositions can be nebulized by use of inert gases for inhalation. Nebulized suspensions may be breathed directly from the nebulizing device or the nebulizing device can be attached to face masks tent, or intermittent positive pressure breathing machine.

The amount of nucleic acid segment that is combined with one or more pharmaceutically acceptable carriers to produce a single dosage form will necessarily vary depending upon the subject treated and the particular route of administration. For further information on routes of administration and dosage regimes the reader is referred to Chapter 25.3 in Volume 5 of Comprehensive Medicinal Chemistry (Corwin Hansch; Chairman of Editorial Board), Pergamon Press 1990.

In one embodiment, the present disclosure provides a method for administering pharmaceutical compositions comprising a plurality of lipid nanoparticles comprising the compound of Formula (I), or a pharmaceutically acceptable salt thereof, and a therapeutically effective amount of a nucleic acid segment in a subject in need thereof.

The term “subject” includes warm-blooded mammals, for example, primates, cows, pigs, sheep, dogs, cats, rabbits, rats, and mice. In some embodiments, the subject is a primate, for example, a human. In some embodiments, the subject is in need of treatment (e.g., the subject would benefit biologically or medically from treatment).

The lipid nanoparticles disclosed herein may further serve as drug delivery vehicles for selective delivery of nucleic acid segments to target cells and tissues, such as antisense oligonucleotides, DNA, mRNAs, siRNAs, Cas9-guideRNA complex. Thus, in one embodiment, is a method of delivering a nucleic acid segment to a cell comprising contacting the cell, in vitro or in vivo, with a pharmaceutical composition comprising a plurality of lipid nanoparticles comprising the compound of Formula (I), or a pharmaceutically acceptable salt thereof, and a therapeutically effective amount of a nucleic acid segment. In some embodiments, the nucleic acid segment modulates expression, for example, by increasing or decreasing expression, or by upregulating or downregulating expression of the polypeptide.

Another embodiment provides a method for delivering a therapeutically effective amount of a nucleic acid segment to a subject in need thereof, comprising administering to the subject a pharmaceutical composition comprising a plurality of lipid nanoparticles comprising the compound of Formula (I), or a pharmaceutically acceptable salt thereof, and a therapeutically effective amount of a nucleic acid segment.

The pharmaceutical compositions comprising a plurality of lipid nanoparticles comprising the compound of Formula (I), or a pharmaceutically acceptable salt thereof, and a nucleic acid segment disclosed herein may be used to treat a wide variety of disorders and diseases characterized by underexpression of a polypeptide in a subject, overexpression of a polypeptide in a subject, and/or absence/presence of a polypeptide in a subject. Accordingly, disclosed are methods of treating a subject suffering from a disease or disorder comprising administering to the subject a pharmaceutical composition comprising a plurality of lipid nanoparticles comprising the compound of Formula (I), or a pharmaceutically acceptable salt thereof, and a therapeutically effective amount of a nucleic acid segment.

Further disclosed is the use of a pharmaceutical composition comprising a plurality of lipid nanoparticles comprising the compound of Formula (I), or a pharmaceutically acceptable salt thereof, and a therapeutically effective amount of a nucleic acid segment, to treat a disease or disorder.

Further disclosed is a pharmaceutical composition for use in the treatment of a disease or disorder, wherein the pharmaceutical composition comprises a plurality of lipid nanoparticles comprising the compound of Formula (I), or a pharmaceutically acceptable salt thereof, and a therapeutically effective amount of a nucleic acid segment.

Further disclosed are methods for modulating protein expression in cells, comprising administering a pharmaceutical composition comprising a plurality of lipid nanoparticles comprising the compound of Formula (I), or a pharmaceutically acceptable salt thereof, and a nucleic acid segment to a subject in need thereof. In at least one embodiment, protein expression may be increased by a factor of about 2 up to 24 hours. In another embodiment, protein expression may be increased by a factor of about 3 up to 72 hours.

### EXAMPLES

### General Methods

¹H NMR: 300 MHz; probe: 5 mm broadband liquid probe BBFO with ATM+Z PABBO BB-1H/D; magnet: ULTRASHIELD™ 300; Crate: AVANCE Ill 300; Auto Sampler: SampleXpress™ 60; software: Topspin 3. 400 MHz; probe: 5 mm Broadband liquid probe BBFO with ATM+Z PABBO BB-1H/D; magnet ASCEND™ 400; crate AVANCE Ill 300; auto sampler SampleXpress™ 60; software: Topspin 3. All spectra were calibrated with TMS as internal reference.

¹H NMR: 500 MHz; probe: 5 mm Bruker Smart probe with ATM+Z PABBO 500S1-BBF-H-D; magnet: ASCEND™ 500; Console: AVANCE Neo 500; Auto Sampler: SampleXpress™ 60; software: Topspin 4. Proton chemical shifts are expressed in parts per million (ppm, 6 scale) and are referenced to residual protium in the NMR solvent (Chloroform-d: δ 7.26, Methanol-d4: δ 3.31, DMSO-d6: δ 2.50). Data are represented as follows: chemical shift, multiplicity (s=singlet, d=doublet, t=triplet, q=quartet, dd=doublet of doublets, dt=doublet of triplets, m=multiplet, br=broad, app=apparent), integration, and coupling constant (J) in Hertz (Hz).

LCMS: Instrument Shimadzu LCMS-2020 coupled with DAD detector, ELSD detector and 2020EV MS; column Shim-pack XR-ODS C18 (50×3.0 mm, 2.2 μm); eluent A water (0.05% TFA), eluent B MeCN (0.05% TFA); gradient 5-95% B in 2.00 min, hold 0.70 min (method A) or 60-95% B in 1.00 min, hold 1.70 min (method B); flow 1.20 mL/min; PDA detection (SPD-M20A) 190-400 nm. Mass spectrometer in ESI mode.

UPLC-MS was carried out using a Waters Acquity UPLC and Waters SQD mass spectrometer (column temp 30° C., UV detection=210-400 nm, mass spec=ESI with positive/negative switching) at a flow rate of 1 mL/min using a solvent gradient of 2 to 98% B over 1.5 mins (total runtime with equilibration back to starting conditions 2 min), where A=0.1% formic acid in water and B=0.1% formic acid in acetonitrile (for acid work) or A=0.1% ammonium hydroxide in water and B=acetonitrile (for base work). For acid analysis the column used was Waters Acquity HSS T3, 1.8 mm, 2.1×30 mm, for base analysis the column used was Waters Acquity BEH C18, 1.7 mm, 2.1×30 mm.

HPLC: Instrument Shimadzu LCMS-2020 coupled with a DAD detector, CAD detector; column Ascentis Express C18 (100×4.6 mm), 2.7 μm; mobile phase A water (0.05% TFA), mobile phase B MeCN; gradient 10-95% B in 4.00 min, hold 8 min, or as indicated, flow 1.50 mL/min; purity as area %.

Preparative HPLC: instruments Waters 2545 Binary Gradient module, Waters 2767 Sample Manager, Waters 2489 UV/Visible Detector, Waters SQ Detector 2. Method A: column XSelect CSH Prep C18 OBD Column, 19×250 mm, 5 μm; mobile phase A water (0.05% TFA), mobile phase B MeCN; flow rate 25 mL/min, gradient as indicated. Method B: column SunFire C18 OBD, 19×250 mm, 5 μm; mobile phase A water (0.05% TFA), mobile phase B MeCN; flow rate 60 mL/min, gradient as indicated.

### Abbreviations

1,2-DCE 1,2-dichloroethane
ACN acetonitrile
CPME cyclopentyl methyl ether
DCM dichloromethane
DMSO dimethylsulfoxide
DIEA N,N-diisopropylethylamine
DIPEA N,N-diisopropylethylamine
DMAP N,N-dimethylaminopyridine
<d>_{N} number-average particle diameter
<d>_{z} z-average particle diameter
EDC 1-Ethyl-3-(3-dimethylaminopropyl)carbodiimide
EDCl 1-Ethyl-3-(3-dimethylaminopropyl)carbodiimide
EE % encapsulation efficacy
EtOAc ethyl acetate
HPLC High-performance liquid chromatography
KI potassium iodide
MC3 (6Z,9Z,28Z,31Z)-heptatriacont-6,9,28,31-tetraene-19-yl 4-(dimethylamino)butanoate
NMP N-methyl-2-pyrrolidone
PDI polydispersity index
PE petrolether (30-50)
PBS phosphate-buffered saline
rt room temperature
TEA triethylamine
THF tetrahydrofuran
UPLC Ultra-performance liquid chromatography
Z-pot zeta-potential

### Example 1. Synthesis of Compound 1

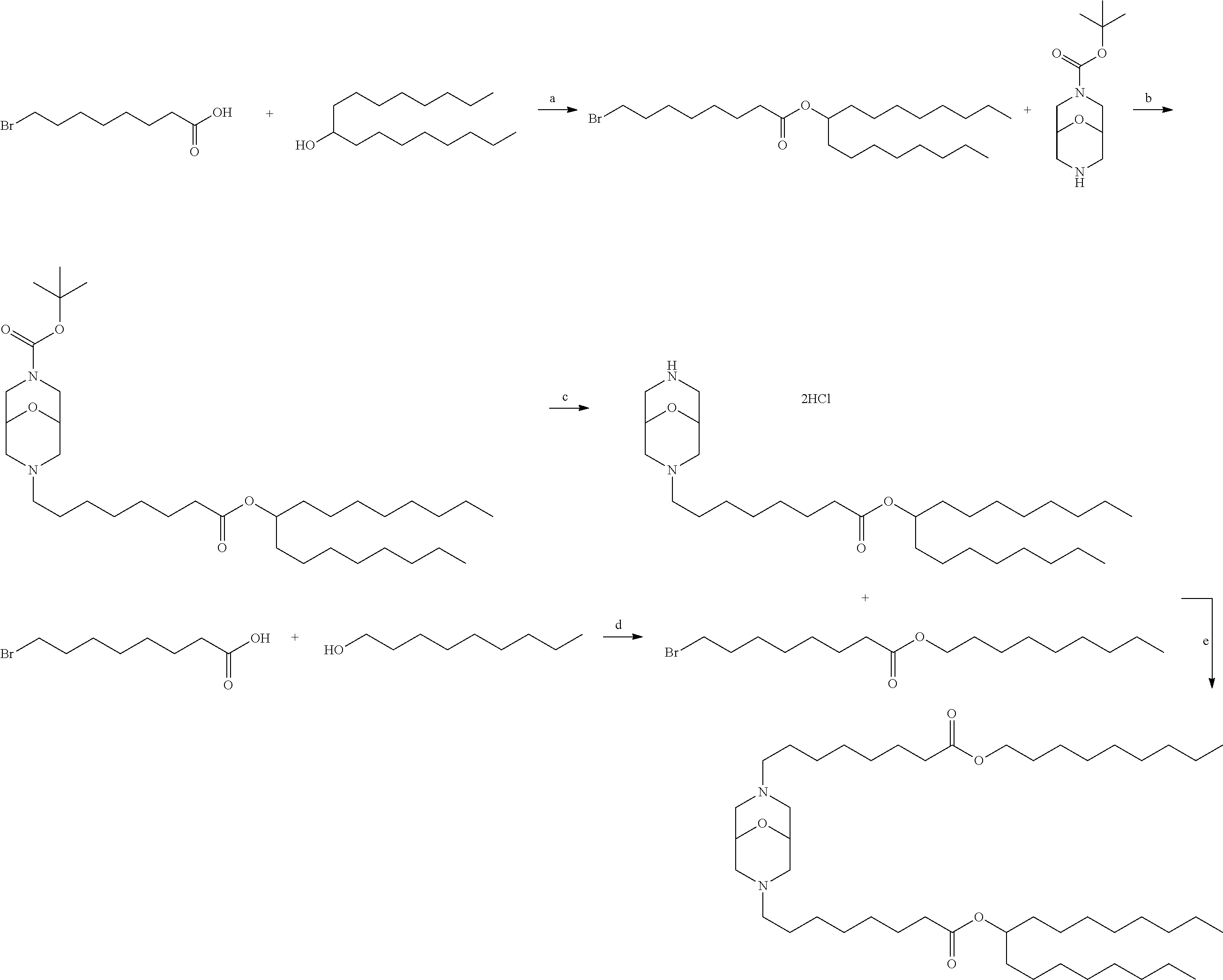

Reagents. a) EDC, DIPEA, DMAP, DCM; b) K₂CO₃, KI, ACN; c) 4M HCl, dioxane; d) EDC, DIPEA, DMAP, DCM; e) DIPEA, KI, ACN

### Intermediate 1: Heptadecan-9-yl 8-bromooctanoate

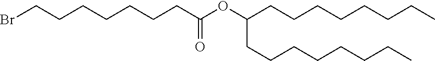 [SMILES: CCCCCCCCC(CCCCCCCC)OC(=O)CCCCCCCBr]

### Step a:

EDC (0.785 g, 4.09 mmol) was added in one portion to a stirred mixture of 8-bromooctanoic acid (0.522 g, 2.34 mmol), heptadecan-9-ol (0.5 g, 1.95 mmol), DMAP (0.048 g, 0.39 mmol), and DIPEA (1.396 ml, 7.99 mmol) in DCM (15 mL) under argon. The resulting mixture was stirred at room temperature for 16 hours. The reaction mixture was concentrated under reduced pressure to dryness and redissolved in EtOAc (50 mL), and washed sequentially with saturated aqueous NaHCO₃ (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 20% EtOAc in hexanes. Product fractions were concentrated under reduced pressure to dryness to afford heptadecan-9-yl 8-bromooctanoate (0.714 g, 79%) as a colorless oil. 1H NMR (500 MHz, CHLOROFORM-d, 27° C.) 0.89 (6H, t), 1.27-1.70 (36H, m), 1.77-1.94 (2H, m), 2.21-2.37 (2H, t), 3.41 (2H, t), 4.78-4.95 (1H, m).

### Intermediate 2: Tert-butyl 7-(8-(heptadecan-9-yloxy)-8-oxooctyl)-9-oxa-3,7-diazabicyclo[3.3.1]nonane-3-carboxylate

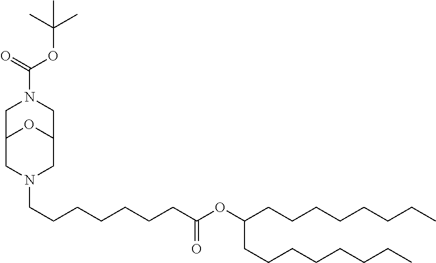 [SMILES: CCCCCCCCC(CCCCCCCC)OC(=O)CCCCCCCN1CC2CN(C(=O)OC(C)(C)C)CC(C1)O2]

### Step b:

Heptadecan-9-yl 8-bromooctanoate (0.808 g, 1.75 mmol) (Intermediate 1) was added dropwise to a stirred mixture of tert-butyl 9-oxa-3,7-diazabicyclo[3.3.1]nonane-3-carboxylate hydrochloride (0.386 g, 1.46 mmol), potassium carbonate (0.423 g, 3.06 mmol) and potassium iodide (0.048 g, 0.29 mmol) in acetonitrile (10 mL) under argon. The resulting mixture was stirred at 80° C. for 16 hours. The reaction mixture was concentrated under reduced pressure to dryness and redissolved in EtOAc (100 mL), and washed sequentially with saturated aqueous Na2CO3 (100 mL) and saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford tert-butyl 7-(8-(heptadecan-9-yloxy)-8-oxooctyl)-9-oxa-3,7-diazabicyclo[3.3.1]nonane-3-carboxylate (0.747 g, 84%) as a pale yellow oil. 1H NMR (500 MHz, CHLOROFORM-d, 22° C.) 0.88 (6H, t), 1.20-1.67 (47H, m), 2.02-2.19 (2H, m), 2.22-2.31 (2H, t), 2.35-2.49 (2H, m), 2.77-2.96 (2H, m), 3.18-3.38 (2H, m), 3.68-3.83 (2H, m), 3.92-4.17 (2H, m), 4.87 (1H, m).

### Intermediate 3: Heptadecan-9-yl 8-(9-oxa-3,7-diazabicyclo[3.3.1]nonan-3-yl)octanoate dihydrochloride

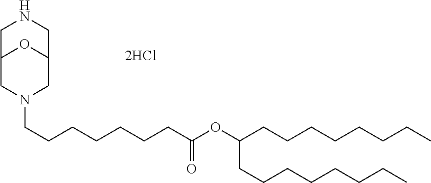 [SMILES: CCCCCCCCC(CCCCCCCC)OC(=O)CCCCCCCN1CC2CNCC(C1)O2]

### Step c:

HCl (2.250 ml, 9.00 mmol) in dioxane was added dropwise to a stirred mixture of tert-butyl 7-(8-(heptadecan-9-yloxy)-8-oxooctyl)-9-oxa-3,7-diazabicyclo[3.3.1]nonane-3-carboxylate (0.548 g, 0.90 mmol) (Intermediate 2) in dioxane (10 mL) at 0° C. under argon. The resulting mixture was warmed and stirred at room temperature for 4 hours. The reaction was concentrated under reduced pressure to dryness and washed with dioxane (3×50 mL) to afford heptadecan-9-yl 8-(9-oxa-3,7-diazabicyclo[3.3.1]nonan-3-yl)octanoate dihydrochloride (0.472 g, 96%) as a pale yellow oil. 1H NMR (500 MHz, METHANOL-d4, 27° C.) 0.90 (6H, t), 1.29 (38H, m), 2.24-2.39 (2H, t), 3.49-3.59 (3H, t), 4.85-4.91 (1H, m).

### Intermediate 4: Nonyl 8-bromooctanoate

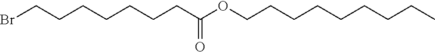 [SMILES: CCCCCCCCCOC(=O)CCCCCCCBr]

### Step d:

EDC (2.79 g, 14.56 mmol) was added in one portion to a stirred solution of 8-bromooctanoic acid (2.320 g, 10.40 mmol), nonan-1-ol (1.205 ml, 6.93 mmol), DMAP (0.169 g, 1.39 mmol), and DIPEA (2.54 ml, 14.56 mmol) in DCM (15 mL). The resulting mixture was stirred at room temperature for 16 hours. The reaction mixture was concentrated under reduced pressure to dryness and redissolved in EtOAc (100 mL), and washed sequentially with saturated aqueous NaHCO₃ (100 mL) and saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 20% EtOAc in hexanes. Product fractions were concentrated under reduced pressure to dryness to afford nonyl 8-bromooctanoate (1.580 g, 65.2%) as a colorless oil. 1H NMR (500 MHz, CHLOROFORM-d, 27° C.) 0.82-0.95 (3H, t), 1.23-1.51 (18H, m), 1.56-1.69 (4H, m), 1.80-1.92 (2H, m), 2.30 (2H, t), 3.33-3.48 (2H, t), 4.07 (2H, t).

### Compound 1: Heptadecan-9-yl 8-(7-(8-(nonyloxy)-8-oxooctyl)-9-oxa-3,7-diazabicyclo[3.3.1]nonan-3-yl)octanoate

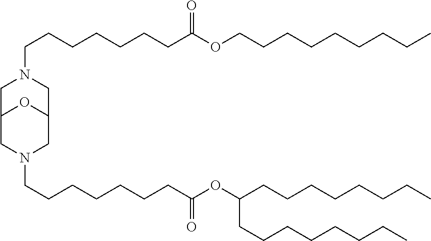 [SMILES: CCCCCCCCCOC(=O)CCCCCCCN1CC2CN(CCCCCCCC(=O)OC(CCCCCCCC)CCCCCCCC)CC(C1)O2]

### Step e:

Nonyl 8-bromooctanoate (0.144 g, 0.41 mmol) (Intermediate 4) was added dropwise to a stirred mixture of heptadecan-9-yl 8-(9-oxa-3,7-diazabicyclo[3.3.1]nonan-3-yl)octanoate dihydrochloride (0.2 g, 0.34 mmol) (Intermediate 3) and DIPEA (0.486 ml, 2.78 mmol) in acetonitrile (5 mL) under argon. The resulting mixture was stirred at 80° C. for 16 hours. The reaction mixture was concentrated under reduced pressure to dryness and redissolved in EtOAc (50 mL), and washed sequentially with saturated aqueous NaHCO₃ (50 mL) and saturated aqueous NaCl (50 mL). The organic layer was dried over Na₂SO₄, filtered and concentrated under reduced pressure to dryness to afford crude product. The resulting residue was purified twice by flash silica chromatography, elution gradient 0 to 100% (20% MeOH and 1% NH₄OH in DCM) in DCM. Product fractions were concentrated under reduced pressure to dryness to afford heptadecan-9-yl 8-(7-(8-(nonyloxy)-8-oxooctyl)-9-oxa-3,7-diazabicyclo[3.3.1]nonan-3-yl)octanoate (0.119 g, 44.5%) as a pale yellow oil. 1H NMR (500 MHz, METHANOL-d4, 27° C.) 0.90 (9H, t), 1.23-1.43 (48H, m), 1.48-1.68 (14H, m), 2.24-2.34 (8H, m), 2.41-2.52 (4H, br d), 2.87 (4H, br d), 3.83-3.92 (2H, m), 4.07 (2H, s), 4.88 (1H, m). UPLC, ms detection (ES+) ([M+H]+)=777.8 Da; RT ELSD=1.91 min.

### Example 2. Synthesis of Compound 2

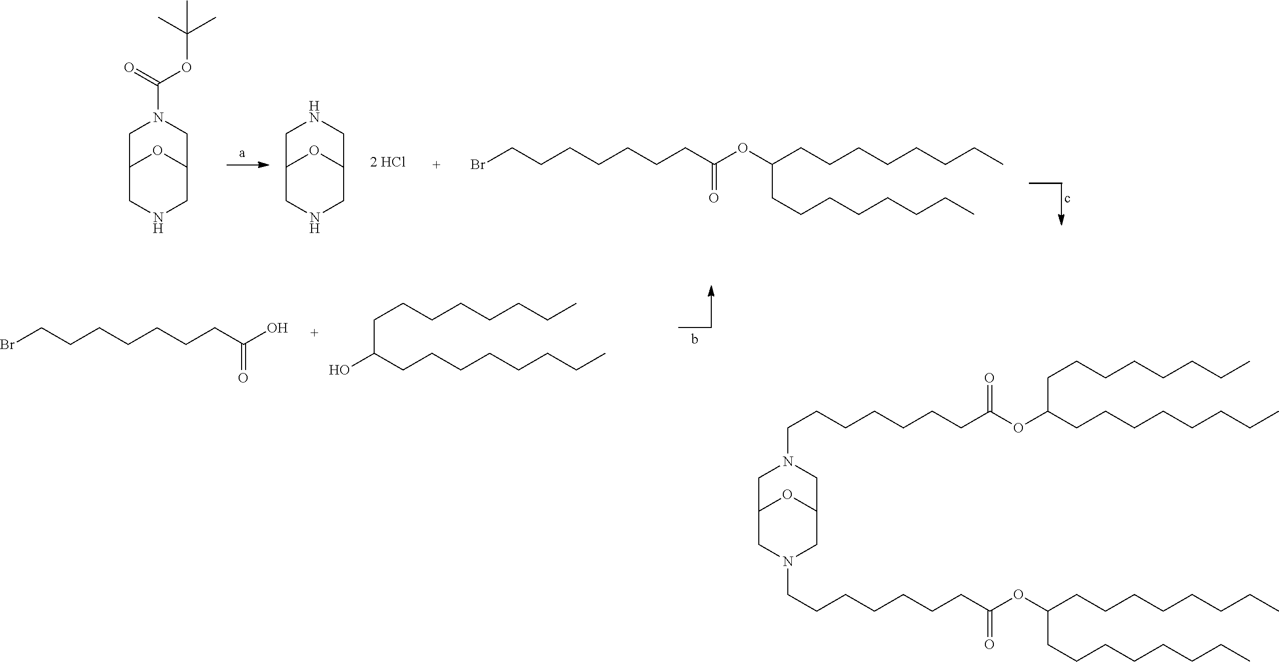

Reagents. a) 4M HCl, dioxane; b) EDC, DIPEA, DMAP, DCM; c) DIPEA, KI, ACN

### Intermediate 1: 9-oxa-3,7-diazabicyclo[3.3.1]nonane dihydrochloride

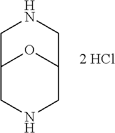

### Step a:

HCl (2 ml, 65.83 mmol) was added dropwise to a stirred mixture of tert-butyl 9-oxa-3,7-diazabicyclo[3.3.1]nonane-3-carboxylate hydrochloride (0.3 g, 1.13 mmol) in 1,4-dioxane (8 mL) under argon. The reaction was stirred overnight at room temperature until precipitate formed. The reaction mixture was concentrated under reduced pressure to dryness to afford to afford 9-oxa-3,7-diazabicyclo[3.3.1]nonane (0.220 g, 97%) as a white powder. 1H NMR (500 MHz, METHANOL-d4, 27° C.) 3.51 (8H, m), 4.41 (2H, br t).

### Intermediate 2: Heptadecan-9-yl 8-bromooctanoate

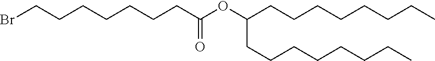 [SMILES: CCCCCCCCC(CCCCCCCC)OC(=O)CCCCCCCBr]

### Step b:

EDC (0.785 g, 4.09 mmol) was added in one portion to a stirred mixture of 8-bromooctanoic acid (0.522 g, 2.34 mmol), heptadecan-9-ol (0.5 g, 1.95 mmol), DMAP (0.048 g, 0.39 mmol), and DIPEA (1.396 ml, 7.99 mmol) in DCM (15 mL) under argon. The resulting mixture was stirred at room temperature for 16 hours. The reaction mixture was concentrated under reduced pressure to dryness and redissolved in EtOAc (50 mL), and washed sequentially with saturated aqueous NaHCO₃ (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 20% EtOAc in hexanes. Product fractions were concentrated under reduced pressure to dryness to afford heptadecan-9-yl 8-bromooctanoate (0.714 g, 79%) as a colorless oil. 1H NMR (500 MHz, CHLOROFORM-d, 27° C.) 0.89 (6H, t), 1.27-1.70 (36H, m), 1.77-1.94 (2H, m), 2.21-2.37 (2H, t), 3.41 (2H, t), 4.78-4.95 (1H, m).

### Compound 2: Di(heptadecan-9-yl) 8,8′-(9-oxa-3,7-diazabicyclo[3.3.1]nonane-3,7-diyl)dioctanoate

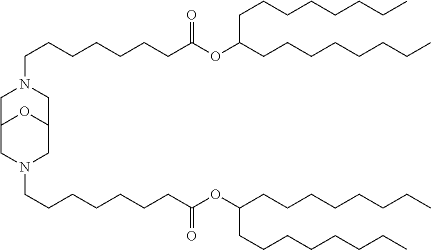 [SMILES: CCCCCCCCC(CCCCCCCC)OC(=O)CCCCCCCN1CC2CN(CCCCCCCC(=O)OC(CCCCCCCC)CCCCCCCC)CC(C1)O2]

### Step c:

Heptadecan-9-yl 8-bromooctanoate (0.285 g, 0.62 mmol) (Intermediate 2) was added dropwise to a stirred mixture of 9-oxa-3,7-diazabicyclo[3.3.1]nonane dihydrochloride (0.04 g, 0.20 mmol) (Intermediate 1), DIPEA (0.142 ml, 0.82 mmol), and potassium iodide (6.60 mg, 0.04 mmol) in acetonitrile (5 mL) at 25° C. under argon. The resulting mixture was stirred at 80° C. for 16 hours. The reaction mixture was concentrated under reduced pressure to dryness and redissolved in EtOAc (50 mL), and washed sequentially with saturated aqueous Na2CO3 (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford di(heptadecan-9-yl) 8,8′-(9-oxa-3,7-diazabicyclo[3.3.1]nonane-3,7-diyl)dioctanoate (0.062 g, 34.9%) as a pale yellow oil. 1H NMR (500 MHz, CHLOROFORM-d, 27° C.) 0.89 (12H, t), 1.15-1.72 (76H, m), 2.28 (8H, m), 2.41-2.50 (4H, m), 2.76-2.84 (4H, m), 3.88 (2H, br s), 4.87 (2H, m). UPLC, ms detection (ES+) ([M+H]+)=890.2; RT ELSD=2.27 min.

### Example 3. Synthesis of Compound 3

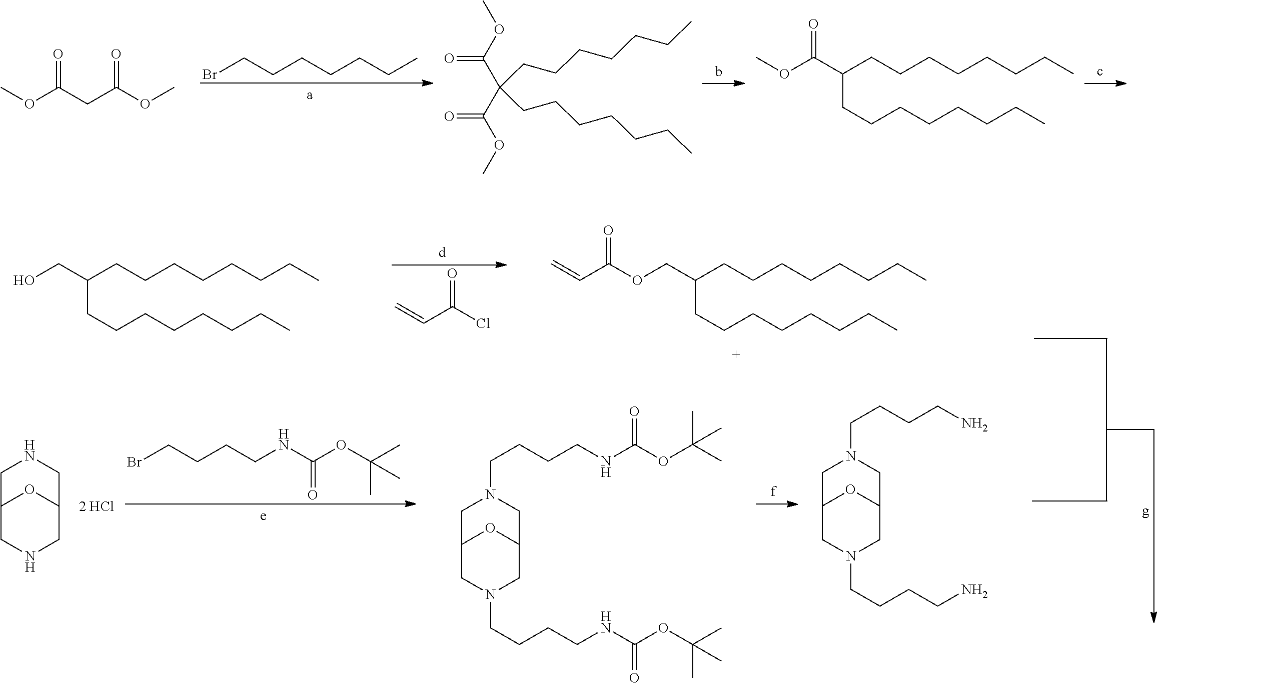

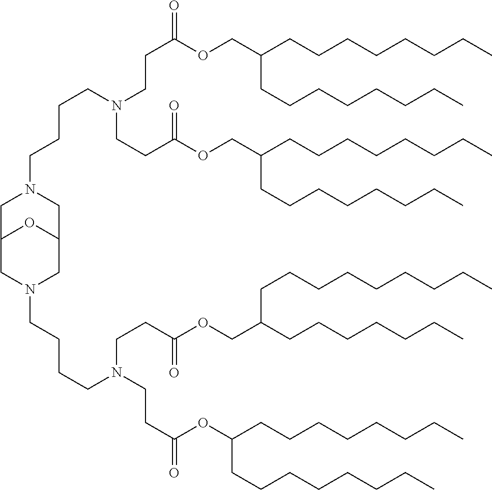 [SMILES: CCCCCCCCCC(CCCCCCC)COC(=O)CCN(CCCCN1CC2CN(CCCCN(CCC(=O)OCC(CCCCCCCC)CCCCCCCC)CCC(=O)OCC(CCCCCCCC)CCCCCCCC)CC(C1)O2)CCC(=O)OC(CCCCCCCC)CCCCCCCC]

Reagents. a) NaH, DMF; b) LiCl, H₂O, DMSO; c) LiAlH₄, THF; d) Acryloyl Chloride, TEA, DCM; e) DIPEA, KI, ACN; f) HCl, dioxane; g) TEA, IPA.

### Intermediate 1: Dimethyl 2,2-dioctylmalonate

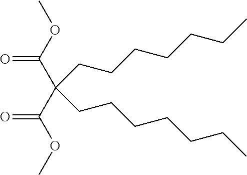 [SMILES: CCCCCCCC(CCCCCCC)(C(=O)OC)C(=O)OC]

### Step a:

Sodium hydride (3.48 g, 87.05 mmol) was slowly suspended in dry DMF under nitrogen and the mixture was cooled to 0° C. dimethyl malonate (4.33 ml, 37.85 mmol) and 1-bromooctane (19.61 ml, 113.54 mmol) in DMF (25 mL each) was added one after another and the mixture was stirred at room temperature for 3 h. After reaction, water (250 mL) was added and the aqueous layer was extracted with (Et₂O) (3×100 mL). The combined organic layers were washed sequentially with water (100 mL) and saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 50% EtOAc in hexanes. Product fractions were concentrated under reduced pressure to dryness to afford dimethyl 2,2-dioctylmalonate (8.89 g, 65.9%) as a pale yellow oil. 1H NMR (500 MHz, CHLOROFORM-d, 27° C.) 0.85-0.92 (6H, 3), 1.27 (24H, m), 1.77-1.93 (4H, m), 3.62-3.77 (6H, s).

### Intermediate 2; Methyl 2-octyldecanoate

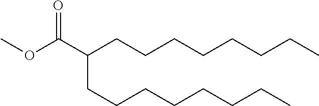 [SMILES: CCCCCCCCC(CCCCCCCC)C(=O)OC]

### Step b:

A mixture of dimethyl 2,2-dioctylmalonate (9.2 g, 25.80 mmol) (Intermediate 1), lithium chloride (1.422 g, 33.54 mmol), and water (0.604 g, 33.54 mmol) in DMSO (80 mL) was stirred at reflux for 24 h. After cooling to room temperature, water (150 mL) was added to quench the reaction. The reaction mixture was extracted with (Et₂O) (3×50 mL). The combined organic layers were washed sequentially with water (3×50 mL). The organic layer was dried over Na₂SO₄, filtered and concentrated under reduced pressure to dryness to afford crude product. The resulting residue was purified by flash silica chromatography, elution gradient 0 to 50% EtOAc in hexanes. Product fractions were concentrated under reduced pressure to dryness to afford methyl 2-octyldecanoate (7.30 g, 95%) as a yellow liquid. 1H NMR (500 MHz, CHLOROFORM-d, 27° C.) 0.89 (6H, t), 1.18-1.71 (28H, m), 2.28-2.39 (1H, m), 3.68 (3H, s).

### Intermediate 3: 2-octyldecan-1-ol

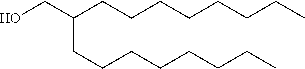 [SMILES: CCCCCCCCC(CO)CCCCCCCC]

### Step c:

To a solution of methyl 2-octyldecanoate (7.3 g, 24.45 mmol) (Intermediate 2) in THF (100 mL), lithium aluminum hydride (14.67 ml, 29.35 mmol) was added dropwise at 0° C. The reaction was then warmed and stirred at room temperature for 24 h. After completion, 3M HCl (50 mL) was added to quench the reaction. The reaction mixture was diluted with water (100 mL) and DCM (100 mL). The layers were separated, and the aqueous layer was extracted with (DCM) (3×50 mL). The combined organic layers were washed with saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 2-octyldecan-1-ol (4.56 g, 68.9%) as a yellow liquid. 1H NMR (500 MHz, CHLOROFORM-d, 27° C.) 0.89 (6H, t), 1.16-1.39 (28H, m), 1.42-1.50 (1H, m), 3.55 (2H, br s).

### Intermediate 4: 2-octyldecyl acrylate

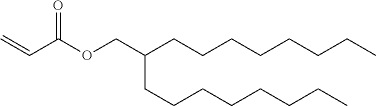 [SMILES: C=CC(=O)OCC(CCCCCCCC)CCCCCCCC]

### Step d:

Acryloyl chloride (0.358 ml, 4.44 mmol) was added dropwise to a stirred mixture of 2-octyldecan-1-ol (1 g, 3.70 mmol) (Intermediate 3) and TEA (2.113 ml, 15.16 mmol) in DCM (20 mL) at 0° C. under argon. The resulting mixture was stirred at room temperature for 16 hours. The reaction mixture was concentrated under reduced pressure and diluted with DCM (100 mL), and washed with saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 100% EtOAc in hexanes. Product fractions were concentrated under reduced pressure to dryness to afford 2-octyldecyl acrylate (0.850 g, 70.8%) as a pale yellow oil. 1H NMR (500 MHz, CHLOROFORM-d, 27° C.) 0.89 (6H, t), 1.23-1.35 (28H, m), 1.61-1.75 (1H, m), 4.07 (2H, d), 5.82 (1H, d), 6.13 (1H, dd), 6.40 (1H, d).

### Intermediate 5: Di-tert-butyl ((9-oxa-3,7-diazabicyclo[3.3.1]nonane-3,7-diyl)bis(butane-4,1-diyl))dicarbamate

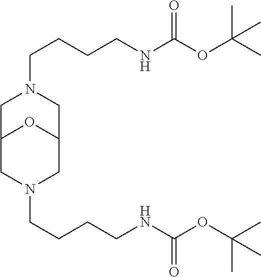 [SMILES: CC(C)(C)OC(=O)NCCCCN1CC2CN(CCCCNC(=O)OC(C)(C)C)CC(C1)O2]

### Step e:

Tert-butyl (4-bromobutyl)carbamate (0.502 g, 1.99 mmol) was added in one portion to a stirred mixture of 9-oxa-3,7-diazabicyclo[3.3.1]nonane dihydrochloride (0.1 g, 0.50 mmol), DIPEA (0.521 ml, 2.98 mmol) and potassium iodide (0.017 g, 0.10 mmol) in acetonitrile (5 mL) under nitrogen. The resulting mixture was stirred at 80° C. for 16 hours. The reaction mixture was concentrated under reduced pressure to dryness and redissolved in EtOAc (50 mL), and washed sequentially with saturated aqueous NaHCO₃ (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford di-tert-butyl ((9-oxa-3,7-diazabicyclo[3.3.1]nonane-3,7-diyl)bis(butane-4,1-diyl))dicarbamate (0.095 g, 40.6%) as a pale yellow oil. 1H NMR (500 MHz, METHANOL-d4) δ ppm 1.4 (18H, s) 1.5-1.6 (8H, m) 2.3 (4H, m) 2.4-2.6 (4H, m) 3.0-3.1 (8H, m) 3.8-3.9 (2H, m) 4.8 (2H, m).

### Intermediate 6: 4,4′-(9-oxa-3,7-diazabicyclo[3.3.1]nonane-3,7-diyl)bis(butan-1-amine) tetrahydrochloride

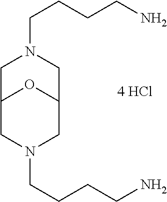 [SMILES: NCCCCN1CC2CN(CCCCN)CC(C1)O2]

### Step f:

A solution of HCl in Dioxane (4M, 10.30 mmol, 2.58 mL) was added to di-tert-butyl ((9-oxa-3,7-diazabicyclo[3.3.1]nonane-3,7-diyl)bis(butane-4,1-diyl))dicarbamate (0.097 g, 0.21 mmol) (Intermediate 5) under inert atmosphere and stirred for 16 hours at room temperature. After rotatory evaporation, the reaction residue was diluted with dioxane (4 mL) and solvent was removed under reduced pressure twice. The solid was concentrated under reduced pressure to dryness to afford to obtain 4,4′-(9-oxa-3,7-diazabicyclo[3.3.1]nonane-3,7-diyl)bis(butan-1-amine) tetrahydrochloride as a white solid. 1H NMR (500 MHz, METHANOL-d4) δ ppm 1.7 (4H, m) 1.7-1.8 (4H, m) 2.7-2.8 (4H, m) 2.9-3.0 (8H, m) 3.3-3.3 (2H, m) 3.5 (4H, br d) 4.1 (2H, br s).

### Compound 3: Tetrakis(2-octyldecyl) 3,3′,3″,3″′-(((9-oxa-3,7-diazabicyclo[3.3.1]nonane-3,7-diyl)bis(butane-4,1-diyl))bis(azanetriyl))tetrapropionate

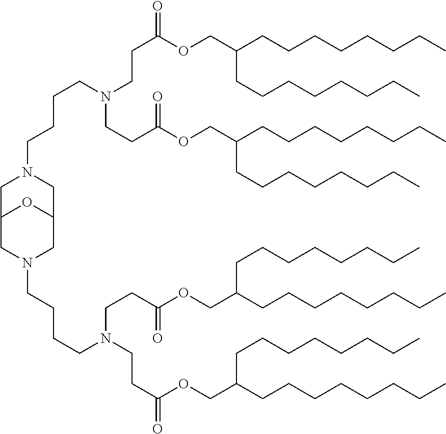 [SMILES: CCCCCCCCC(CCCCCCCC)COC(=O)CCN(CCCCN1CC2CN(CCCCN(CCC(=O)OCC(CCCCCCCC)CCCCCCCC)CCC(=O)OCC(CCCCCCCC)CCCCCCCC)CC(C1)O2)CCC(=O)OCC(CCCCCCCC)CCCCCCCC]

### Step g:

2-octyldecyl acrylate (0.468 g, 1.44 mmol) (Intermediate 4) was added in one portion to a stirred mixture of 4,4′-(9-oxa-3,7-diazabicyclo[3.3.1]nonane-3,7-diyl)bis(butan-1-amine) tetrahydrochloride (0.05 g, 0.12 mmol) (Intermediate 5) and TEA (0.134 ml, 0.96 mmol) in iPrOH (4 mL). The resulting mixture was stirred at 80° C. for 3 days. The reaction mixture was concentrated under reduced pressure to dryness and redissolved in EtOAc (50 mL), and washed sequentially with saturated aqueous NaHCO₃ (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 100% (20% MeOH and NH₄OH in DCM) in DCM. Product fractions were concentrated under reduced pressure to dryness to afford tetrakis(2-octyldecyl) 3,3′,3″,3″′-(((9-oxa-3,7-diazabicyclo[3.3.1]nonane-3,7-diyl)bis(butane-4,1-diyl))bis(azanetriyl))tetrapropionate (0.027 g, 14.06%) as a orange oil. 1H NMR (500 MHz, METHANOL-d4, 27° C.) 0.84-0.99 (24H, t), 1.21-1.41 (116H, m), 1.46-1.56 (4H, m), 1.66 (8H, m), 2.43-2.52 (10H, m), 2.73-2.82 (10H, m), 2.98-3.08 (4H, m), 3.42-3.52 (4H, m), 4.00 (8H, d), 4.18 (2H, br s). MS Detection (ES+) ([M+H]+)=1568.4.

### Example 4. Synthesis of Compound 4

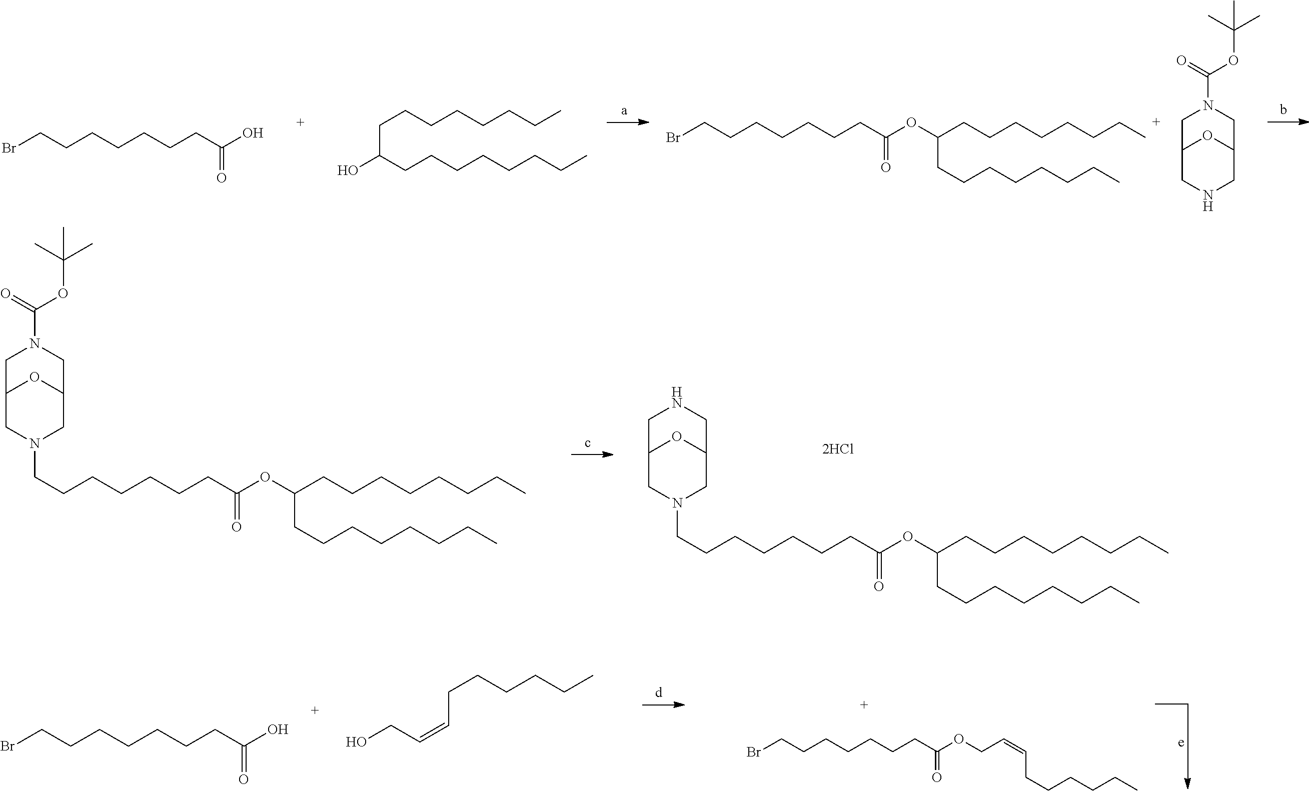

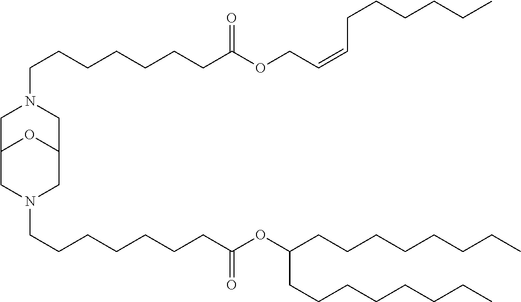 [SMILES: CCCCCC/C=C\COC(=O)CCCCCCCN1CC2CN(CCCCCCCC(=O)OC(CCCCCCCC)CCCCCCCC)CC(C1)O2]

Reagents. a) EDC, DIPEA, DMAP, DCM; b) K₂CO₃, KI, ACN; c) 4M HCl, dioxane; d) EDC, DIPEA, DMAP, DCM; e) DIPEA, KI, ACN

### Intermediate 1: Heptadecan-9-yl 8-bromooctanoate

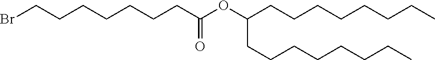 [SMILES: CCCCCCCCC(CCCCCCCC)OC(=O)CCCCCCCBr]

### Step a:

EDC (0.785 g, 4.09 mmol) was added in one portion to a stirred mixture of 8-bromooctanoic acid (0.522 g, 2.34 mmol), heptadecan-9-ol (0.5 g, 1.95 mmol), DMAP (0.048 g, 0.39 mmol), and DIPEA (1.396 ml, 7.99 mmol) in DCM (15 mL) under argon. The resulting mixture was stirred at room temperature for 16 hours. The reaction mixture was concentrated under reduced pressure to dryness and redissolved in EtOAc (50 mL), and washed sequentially with saturated aqueous NaHCO₃ (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 20% EtOAc in hexanes. Product fractions were concentrated under reduced pressure to dryness to afford heptadecan-9-yl 8-bromooctanoate (0.714 g, 79%) as a colorless oil. 1H NMR (500 MHz, CHLOROFORM-d, 27° C.) 0.89 (6H, t), 1.27-1.70 (36H, m), 1.77-1.94 (2H, m), 2.21-2.37 (2H, t), 3.41 (2H, t), 4.78-4.95 (1H, m).

### Intermediate 2: Tert-butyl 7-(8-(heptadecan-9-yloxy)-8-oxooctyl)-9-oxa-3,7-diazabicyclo[3.3.1]nonane-3-carboxylate

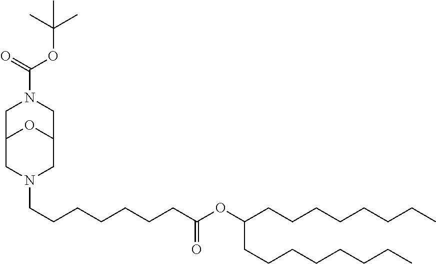 [SMILES: CCCCCCCCC(CCCCCCCC)OC(=O)CCCCCCCN1CC2CN(C(=O)OC(C)(C)C)CC(C1)O2]

### Step b:

Heptadecan-9-yl 8-bromooctanoate (0.808 g, 1.75 mmol) (Intermediate 1) was added dropwise to a stirred mixture of tert-butyl 9-oxa-3,7-diazabicyclo[3.3.1]nonane-3-carboxylate hydrochloride (0.386 g, 1.46 mmol), potassium carbonate (0.423 g, 3.06 mmol) and potassium iodide (0.048 g, 0.29 mmol) in acetonitrile (10 mL) under argon. The resulting mixture was stirred at 80° C. for 16 hours. The reaction mixture was concentrated under reduced pressure to dryness and redissolved in EtOAc (100 mL), and washed sequentially with saturated aqueous Na2CO3 (100 mL) and saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford tert-butyl 7-(8-(heptadecan-9-yloxy)-8-oxooctyl)-9-oxa-3,7-diazabicyclo[3.3.1]nonane-3-carboxylate (0.747 g, 84%) as a pale yellow oil. 1H NMR (500 MHz, CHLOROFORM-d, 22° C.) 0.88 (6H, t), 1.20-1.67 (47H, m), 2.02-2.19 (2H, m), 2.22-2.31 (2H, t), 2.35-2.49 (2H, m), 2.77-2.96 (2H, m), 3.18-3.38 (2H, m), 3.68-3.83 (2H, m), 3.92-4.17 (2H, m), 4.87 (1H, m).

### Intermediate 3: Heptadecan-9-yl 8-(9-oxa-3,7-diazabicyclo[3.3.1]nonan-3-yl)octanoate dihydrochloride

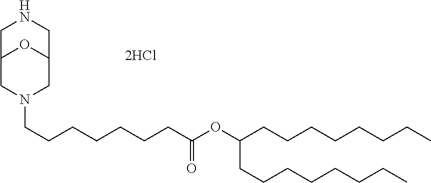 [SMILES: CCCCCCCCC(CCCCCCCC)OC(=O)CCCCCCCN1CC2CNCC(C1)O2]

### Step c:

HCl (2.250 ml, 9.00 mmol) in dioxane was added dropwise to a stirred mixture of tert-butyl 7-(8-(heptadecan-9-yloxy)-8-oxooctyl)-9-oxa-3,7-diazabicyclo[3.3.1]nonane-3-carboxylate (0.548 g, 0.90 mmol) (Intermediate 2) in dioxane (10 mL) at 0° C. under argon. The resulting mixture was warmed and stirred at room temperature for 4 hours. The reaction was concentrated under reduced pressure to dryness and washed with dioxane (3×50 mL) to afford heptadecan-9-yl 8-(9-oxa-3,7-diazabicyclo[3.3.1]nonan-3-yl)octanoate dihydrochloride (0.472 g, 96%) as a pale yellow oil. 1H NMR (500 MHz, METHANOL-d4, 27° C.) 0.90 (6H, t), 1.29 (38H, m), 2.24-2.39 (2H, t), 3.49-3.59 (3H, t), 4.85-4.91 (1H, m).

### Intermediate 4: (Z)-non-2-en-1-yl 8-bromooctanoate

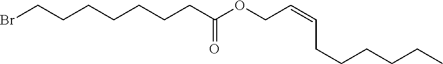 [SMILES: CCCCCC/C=C\COC(=O)CCCCCCCBr]

### Step d:

EDC (2.83 g, 14.76 mmol) was added in one portion to a stirred mixture of 8-bromooctanoic acid (2.353 g, 10.55 mmol), DIPEA (3.68 ml, 21.09 mmol), (Z)-non-2-en-1-ol (1 g, 7.03 mmol), and DMAP (0.172 g, 1.41 mmol) in DCM (15 mL) under argon. The resulting mixture was stirred at room temperature for 16 hours. The reaction mixture was concentrated under reduced pressure to dryness and redissolved in EtOAc (100 mL), and washed sequentially with saturated aqueous NaHCO₃ (100 mL) and saturated aqueous NaCl (100 mL). The organic layer was dried over Na₂SO₄, filtered and concentrated under reduced pressure to dryness to afford crude product. The resulting residue was purified by flash silica chromatography, elution gradient 0 to 100% EtOAc in hexanes. Product fractions were concentrated under reduced pressure to dryness to afford (Z)-non-2-en-1-yl 8-bromooctanoate (1.930 g, 79%) as a pale yellow oil. 1H NMR (500 MHz, CHLOROFORM-d, 27° C.) 0.79-0.95 (3H, t), 1.21-1.50 (14H, m), 1.58-1.69 (2H, m), 1.72-1.93 (2H, m), 2.11 (2H, m), 2.31 (2H, t), 3.31-3.65 (2H, t), 4.63 (2H, d), 5.48-5.73 (2H, m).

### Compound 4: Heptadecan-9-yl (Z)-8-(7-(8-(non-2-en-1-yloxy)-8-oxooctyl)-9-oxa-3,7-diazabicyclo[3.3.1]nonan-3-yl)octanoate

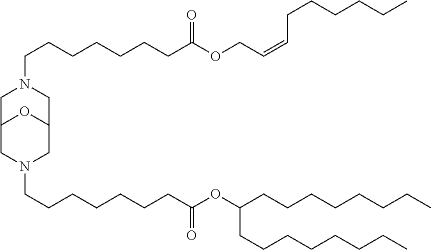 [SMILES: CCCCCC/C=C\COC(=O)CCCCCCCN1CC2CN(CCCCCCCC(=O)OC(CCCCCCCC)CCCCCCCC)CC(C1)O2]

### Step e:

The heptadecan-9-yl 8-(9-oxa-3,7-diazabicyclo[3.3.1]nonan-3-yl)octanoate dihydrochloride (Intermediate 3) dissolved in 1:1 acetonitrile/CPME (10 mL) and DIPEA (0.212 mL, 1.21 mmol) was added at 20° C. under nitrogen. The resulting mixture was stirred at 20° C. for 30 minutes. The (Z)-non-2-en-1-yl 8-bromooctanoate (0.123 g, 0.35 mmol) (Intermediate 4) was added dropwise to the stirred solution under nitrogen. The resulting mixture was heated to 80° C. and stirred over a period of 18 hours under nitrogen. The reaction mixture was concentrated under reduced pressure to dryness and redissolved in EtOAc (50 mL), and washed sequentially with saturated aqueous NaHCO₃ (50 mL) and saturated aqueous sodium chloride (50 mL). The organic layer was dried over Na₂SO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to afford heptadecan-9-yl (Z)-8-(7-(8-(non-2-en-1-yloxy)-8-oxooctyl)-9-oxa-3,7-diazabicyclo[3.3.1]nonan-3-yl)octanoate (0.214 g, 93%) as a pale yellow oil. 1H NMR (500 MHz, METHANOL-d4) 0.86-1.00 (9H, t), 1.23-1.46 (44H, m), 1.56 (8H, m), 1.64 (4H, m), 2.15 (2H, q), 2.24-2.38 (8H, m), 2.51 (4H, br d), 2.92 (4H, br d), 3.91 (2H, br s), 4.64 (2H, d), 4.88-4.93 (1H, m), 5.49-5.71 (2H, m). UPLC, ms detection (ES+) ([M+H]+)=776.0 Da; RT ELSD=1.88 min.

### Example 5. Synthesis of Compound 5

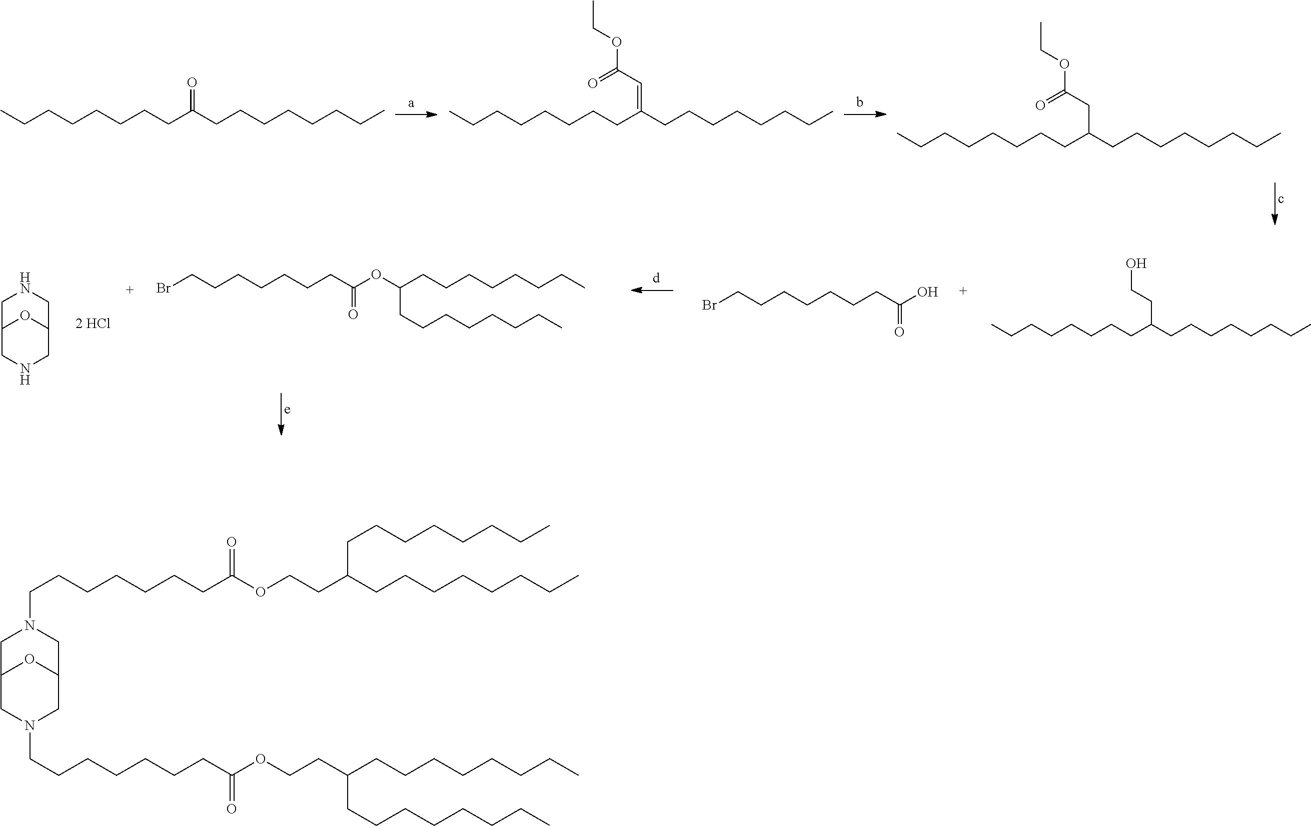

Reagents. a) triethyl phosphonoacetate, NaH, THF; b) Pt(IV)O₂, H₂, CHCl₃/MeOH; c) LiAIH₄, THF; d) EDC, DIPEA, DMAP, DCM; e) DIPEA, KI, ACN

### Intermediate 1: Ethyl 3-octylundec-2-enoate

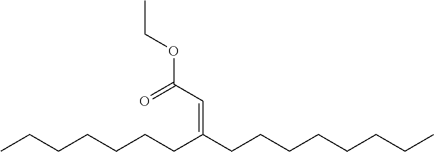 [SMILES: CCCCCCCC/C(=C/C(=O)OCC)CCCCCCC]

### Step a:

Triethyl phosphonoacetate (12.59 ml, 62.88 mmol) was added slowly to a stirred solution of sodium hydride (2.51 g, 62.88 mmol) in THF (50 mL) at 0° C. under nitrogen. The resulting mixture was stirred at 0° C. for 30 minutes. heptadecan-9-one (2 g, 7.86 mmol) was added to the stirred mixture and warmed to 30° C. under nitrogen. The resulting mixture was stirred under reflux for 18 hours. After cooled to room temperature, the reaction mixture was quenched with water (100 mL), extracted with EtOAc (3×50 mL), the combined organic layers were dried over MgSO₄, filtered and concentrated under reduced pressure to dryness to afford yellow oil. The resulting residue was purified by flash silica chromatography, elution gradient 0 to 40% EtOAc in hexanes. 1H NMR was used to determine each fraction's purity. Combined product fractions were concentrated under reduced pressure to dryness to afford ethyl 3-octylundec-2-enoate (1.850 g, 72.5%) as a colorless oil. 1H NMR (500 MHz, CHLOROFORM-d) 0.89 (6H, t), 1.21-1.53 (27H, m), 2.13 (2H, t), 2.53-2.65 (2H, t), 4.15 (2H, q), 5.62 (1H, s).

### Intermediate 2: Ethyl 3-octylundecanoate

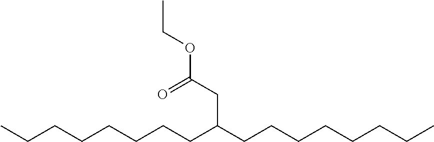 [SMILES: CCCCCCCCC(CCCCCCCC)CC(=O)OCC]

### Step b:

Ethyl 3-octylundec-2-enoate (2.0 g, 6.16 mmol) (Intermediate 1) and platinum(IV) oxide (0.028 g, 0.12 mmol) in CHCl₃/MeOH (5:1) (30 mL) was stirred under a balloon of hydrogen at atmospheric pressure for 16 hours. The reaction mixture was filtered through Celite. The resulting residue was purified by flash silica chromatography, elution gradient 0 to 40% EtOAc in hexanes. Separation was difficult due to overlap with heptadecan-9-one starting material. 1H NMR was used to determine each fraction's purity. Combined product fractions were concentrated under reduced pressure to dryness to afford ethyl 3-octylundecanoate (1.540 g, 77%) as a colorless oil. 1H NMR (500 MHz, CHLOROFORM-d, 27° C.) 0.83-0.95 (6H, t), 1.16-1.39 (31H, m), 1.79-1.92 (1H, br t), 2.22 (2H, d), 4.13 (2H, q).

### Intermediate 3: 3-octylundecan-1-ol

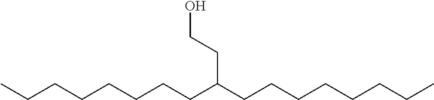 [SMILES: CCCCCCCCC(CCO)CCCCCCCC]

### Step c:

Lithium aluminum hydride (7.75 ml, 7.75 mmol) was added slowly to a stirred solution of ethyl 3-octylundecanoate (2.3 g, 7.04 mmol) (Intermediate 2) in THF (20 mL) at 0° C. under nitrogen. The resulting mixture was stirred at room temperature for 18 hours. After completion, the reaction was cooled to 0° C. and quenched with 3M HCl (100 mL), extracted with EtOAc (3×50 mL), the combined organic layers were dried over MgSO₄, filtered and concentrated under reduced pressure to dryness to afford yellow oil. The resulting residue was purified by flash silica chromatography, elution gradient 0 to 40% EtOAc in hexanes. Product fractions were concentrated under reduced pressure to dryness to afford 3-octylundecan-1-ol (1.354 g, 67.6%) as a colorless oil. 1H NMR (500 MHz, CHLOROFORM-d, 27° C.) 0.89 (6H, t), 1.18-1.36 (28H, m), 1.38-1.46 (1H, br t), 1.53 (2H, q), 3.67 (2H, t).

### Intermediate 4: 3-octylundecyl 8-bromooctanoate

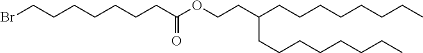 [SMILES: CCCCCCCCC(CCCCCCCC)CCOC(=O)CCCCCCCBr]

### Step d:

EDC (0.707 g, 3.69 mmol) was added in one portion to a stirred mixture of 8-bromooctanoic acid (0.470 g, 2.11 mmol), DIPEA (0.645 ml, 3.69 mmol), 3-octylundecan-1-ol (0.5 g, 1.76 mmol) (Intermediate 3), and DMAP (0.043 g, 0.35 mmol) in DCM (10 mL) under argon. The resulting mixture was stirred at room temperature for 16 hours. The reaction mixture was concentrated under reduced pressure to dryness and redissolved in EtOAc (50 mL), and washed sequentially with saturated aqueous NaHCO₃ (100 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 100% EtOAc in hexanes. Product fractions were concentrated under reduced pressure to dryness to afford 3-octylundecyl 8-bromooctanoate (0.545 g, 63.3%) as a pale yellow oil. 1H NMR (500 MHz, CHLOROFORM-d, 27° C.) 0.83-0.98 (6H, t), 1.20-1.49 (35H, m), 1.53-1.70 (4H, m), 1.79-1.92 (2H, m), 2.30 (2H, t), 3.41 (2H, t), 4.03-4.15 (2H, t).

### Compound 5: Bis(3-octylundecyl) 8,8′-(9-oxa-3,7-diazabicyclo[3.3.1]nonane-3,7-diyl)dioctanoate

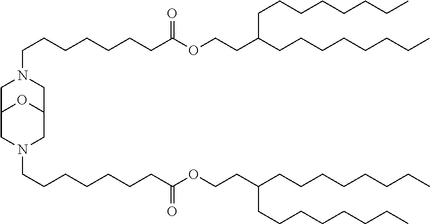 [SMILES: CCCCCCCCC(CCCCCCCC)CCOC(=O)CCCCCCCN1CC2CN(CCCCCCCC(=O)OCCC(CCCCCCCC)CCCCCCCC)CC(C1)O2]

### Step e:

3-octylundecyl 8-bromooctanoate (0.302 g, 0.62 mmol) (Intermediate 4) was added dropwise to a stirred mixture of 9-oxa-3,7-diazabicyclo[3.3.1]nonane dihydrochloride (0.04 g, 0.20 mmol), DIPEA (0.142 ml, 0.82 mmol), and potassium iodide (6.60 mg, 0.04 mmol) in acetonitrile (5 mL) at 25° C. under argon. The resulting mixture was stirred at 80° C. for 16 hours. The reaction mixture was concentrated under reduced pressure to dryness and redissolved in EtOAc (50 mL), and washed sequentially with saturated aqueous Na2CO3 (50 mL) and saturated aqueous NaCl (50 mL). The organic layer was dried over Na₂SO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford bis(3-octylundecyl) 8,8′-(9-oxa-3,7-diazabicyclo[3.3.1]nonane-3,7-diyl)dioctanoate (0.054 g, 28.6%) as a pale yellow oil. 1H NMR (500 MHz, CHLOROFORM-d, 27° C.) 0.89 (12H, t), 1.16-1.69 (80H, m), 1.73-1.85 (2H, m), 2.20-2.26 (4H, br t), 2.26-2.31 (4H, t), 2.40-2.52 (4H, m), 2.82 (4H, br d), 3.82-3.94 (2H, br t), 4.09 (4H, t). UPLC, ms detection (ES+) ([M+H]+)=945.7; RT ELSD=2.43 min.

### Example 6. Preparation of Lipid Nanoparticles (LNP) Formulations Containing eGFP mRNA

LNPs were prepared using a micro-fluidic setup, a NanoAssemblr (Precision NanoSystems Inc.). Briefly, stocks of lipids were dissolved in ethanol and mixed in the appropriate molar ratios to obtain a lipid concentration of 12.5 mM. The size of LNPs was determined by DLS measurements using a Zetasizer Nano ZS from Malvern Instruments Ltd. The number-weighted particle size distributions were calculated using a particle refractive index of 1.45. Characterizations of the LNPs are shown in Table 1. Lipid solution in ethanol (99.5%) was prepared with four different lipid components: ionizable lipid, i.e., MC3, MOD5, Compound 1, Compound 2 or Compound 4; cholesterol (Sigma-Aldrich); DSPC (distearoyl phosphatidyl choline, Avanti Polar Lipids Inc); and a polymer-conjugated lipid. The ratio of lipids in all experiments was ionizable lipid/cholesterol/DSPC/polymer-conjugated lipid (50/38.5/10/1.5 mol %). The total concentration of lipids in all experiments was 12.5 mM. MOD5 is an ionizable lipid with a structure shown below, and more detailed descriptions can be found at Sabnis et al, Mol Therapy, Vol 26, 6, 2018, 1509-1519.

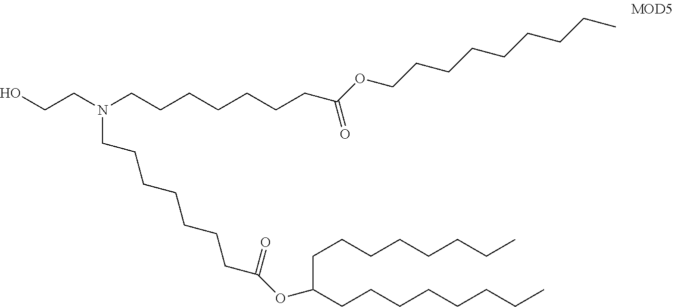 [SMILES: CCCCCCCCCOC(=O)CCCCCCCN(CCO)CCCCCCCC(=O)OC(CCCCCCCC)CCCCCCCC]

A solution of eGFP mRNA (purchased from TriLink Biotechnologies) in citrate buffer was prepared by mixing mRNA dissolved in MilliQ-water, 100 mM citrate buffer (pH 3) and MilliQ-water to give a solution of 50 mM citrate. The mRNA and lipid solutions were mixed in a NanoAssemblr (Precision Nanosystems, Vancouver, BC, Canada) microfluidic mixing system at a mixing ratio of Aq:EtOH=3:1 and a constant flow rate of 12 mL/min. The mRNA in citrate buffer solution was prepared such that, at the time of mixing the ratio between the nitrogen atoms on the ionizable lipid and phosphorus atoms (N/P ratio) on the mRNA chain was either 3:1 or 5:1 (see Table 1). LNPs were dialyzed overnight against 500× sample volume using Slide-A-Lyzer G2 dialysis cassettes from Thermo Scientific with a molecular weight cutoff of 10 K.

The first 0.2-0.35 mL and the last 0.05-0.1 mL of the LNP suspension prepared were discarded while the rest of the volume was collected as the sample fraction. The size of the mRNA lipid nanoparticles was determined by dynamic light scattering measurements using a Zetasizer Nano ZS from Malvern Instruments Ltd, giving directly the z-average particle diameter. The number-weighted particle size distributions and averages were calculated using a particle refractive index of 1.45.

The encapsulation and concentration of mRNA were determined using the Ribo-Green assay. The encapsulation in all of the samples was typically 90-99%. The final mRNA concentration and encapsulation efficiency percentage (% EE) was measured by Quant-it Ribogreen Assay Kit (ThermoFischer Scientific Inc.) using Triton-X100 to disrupt the LNPs. The mRNA encapsulation efficiency was determined according to the following equation:

$$\% EE = [ 1 - ( \frac{non - encapsulated mRNA}{encaspulated mRNA} ) ] \times 100$$

Table 1 summarizes the characterization of LNP formulations comprising MC3, MOD5, Compounds 1, Compound 2 or Compound 4.

TABLE 1
Characterization of LNP Formulations

| | | | Polymer- | | | |
|---|---|---|---|---|---|---|
| | Ionizable | N/P | conjugated | % | <d>_{N} | <d>_{Z} |
| Formulation # | lipid | ratio | lipid | EE | (nm) | (nm) |
| 1 | Compound 1 | 3 | DMPE-PEG2000 | 99 | 40 | 59 |
| 2 | Compound 2 | 5 | DMPE-PEG2000 | 99 | 35 | 50 |
| 3 | Compound 4 | 3 | DMPE-PEG2000 | 97 | 32 | 48 |
| 4 | MC3 | 3 | DMPE-PEG2000 | 99 | 66 | 84 |
| 5 | MOD5 | 3 | DMPE-PEG2000 | 98 | 58 | 76 |

DMPE-PEG2000 is dimyristoyl phosphatidyl ethanolamine-poly(ethylene glycol) 2000 (obtained from NOF Corporation).
DMG-PEG2000 is 1,2-dimyristoyl-rac-glycero-3-methoxypolyethylene glycol-2000.

### Example 7. In Vivo Intratracheal Administration to Rats of LNP-eGFP mRNA Formulations

The in-vivo study was performed at an AAALAC accredited animal facility at AstraZeneca, Gothenburg, Sweden, under approval of the Animal Ethics Committee of Gothenburg (no.82-2015). Male Wistar Han rats were purchased (Charles River Germany Limited) and on arrival were caged in groups of 4 on wood shavings, with diet (R70 Rat and Mouse chow (Lantmsnnen, Stockholm, Sweden) and were provided mains drinking water ad libitum. Animals were approximately 10 weeks of age at the start of dosing. The environment was maintained at a target temperature of 19-24° C. and relative humidity of 40-70%, with a 12-hour light/dark cycle. Animals were acclimatised to the housing conditions for at least 5 days prior to any experimental procedures. Animals that received the inhaled dose were further conditioned to the inhalation restraint procedures for up to 5 days prior to the start of dosing by gradually increasing the duration exposure to restraint procedures up to the maximum expected duration on the respective study.

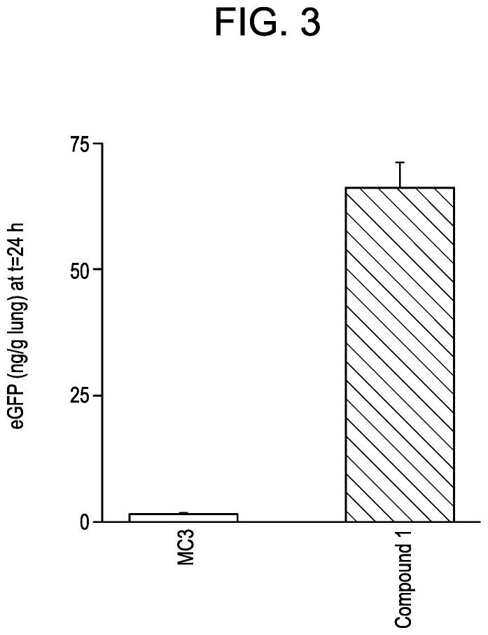

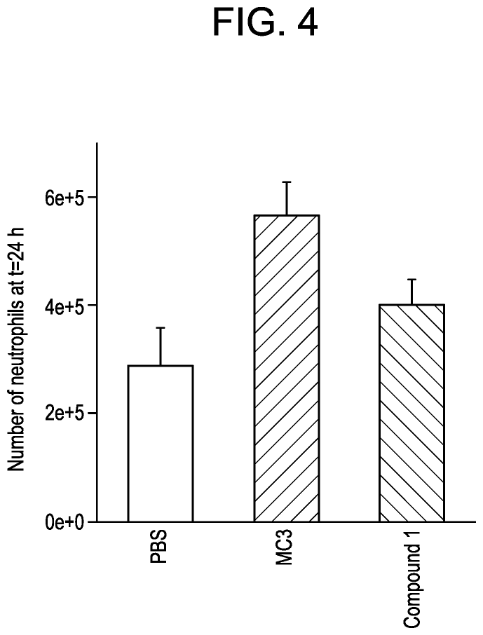

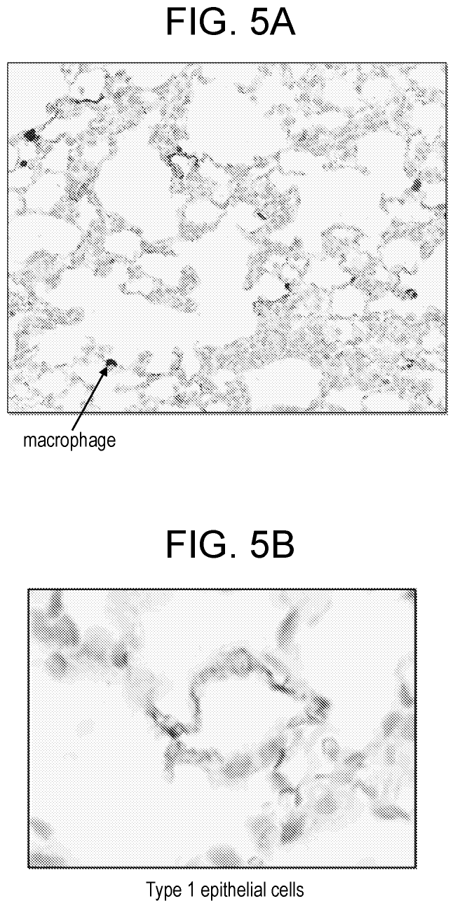

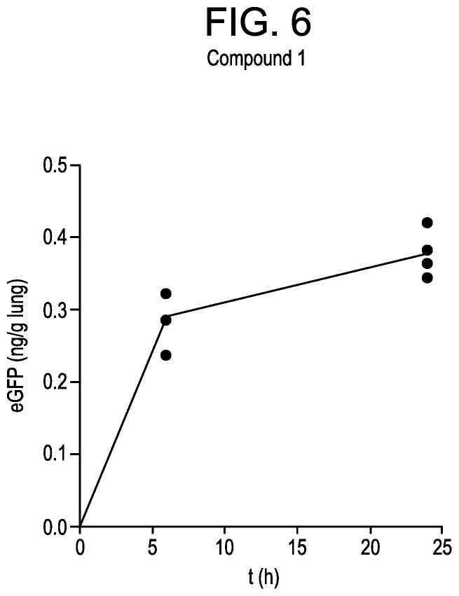

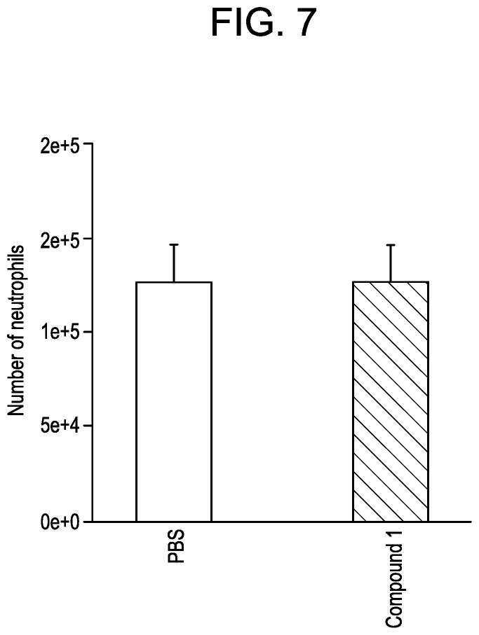

Compound 1 LNP formulation of eGFP mRNA was administered by a single inhaled administration or by a single intratracheal administration at a target lung dose of 0.02 mg/kg eGFP. A group of the same size were used as a placebo control where animals were exposed to Phosphate buffered saline by inhalation or intratracheal installation. The expression levels of eGFP in rats lung at 24 hours after intratracheal administration were shown in FIG. 3, and the BALF neutrophil concentrations in rat BALF at 24 hours after intratracheal administration were shown in FIG. 4. The expression levels of eGFP in rats lung at 5 hours and 24 hours after inhalation administration were shown in FIG. 6, and the BALF neutrophil concentrations in rat BALF at 24 hours after inhalation administration were shown in FIG. 7. Furthermore, immunohistochemistry (IHC) testing indicates the expression of eGFP not only in macrophages but also in type 1 epithelial cells (see FIGS. 5A and 5B).

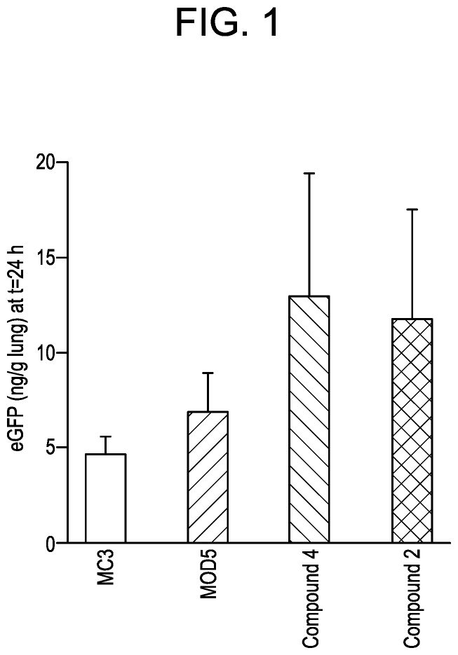

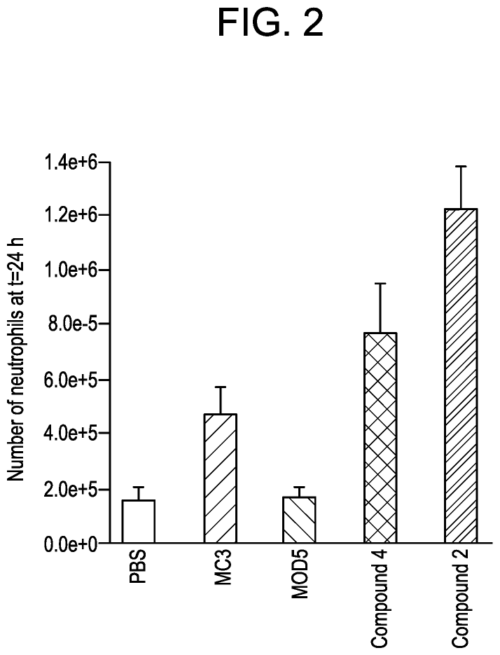

MC3, MOD5, Compound 2 and Compound 4 LNP formulations of eGFP mRNA as described in Example 6 was each administered by a single intratracheal administration at a target lung dose of 0.1 mg/kg eGFP. A group of the same size were used as a placebo control where animals were exposed to Phosphate buffered saline by intratracheal installation. The expression levels of eGFP in rats lung at 24 hours were shown in FIG. 1, and the BALF neutrophil concentrations in rat BALF at 24 hours were shown in FIG. 2.

### Inhalation Dosing

The aerosol was generated using an Aerogen Solo vibrating mesh nebuliser (Galway Ireland). The nebuliser was filled with 5.6 mL of the vehicle or the eGFP mRNA in LNP formulation and was nebulised at ca 60 μl/min for the duration of the dosing. The rats were place into a rodent restraint and then placed on an AstraZeneca designed inhalation dosing system. The animals were monitored through the experimental procedure for any signs of ill effect following the dosing with no clinical observations or abnormal change in body weight noted. At 24 hours after final dose the rats were terminated by sedation of isoflurane anesthesia and then cutting of vena cava and removal of the heart.

### Intratracheal Dosing

Rats were be anaesthetized with Isoflurane mixture (air/oxygen and 3,5% isoflurane), put in a supine position with 30-40° angle and then instilled with eGFP mRNA in LNP formulation or vehicle using a modified metal cannula with bolus-bulb on the top. After the dosing, rats will be placed in cages in a supine position with the head up until regained consciousness. The animals were monitored through the experimental procedure for any signs of ill effect following the dosing with no clinical observations or abnormal change in body weight noted. At 24 hours after final dose the rats were terminated by sedation of isoflurane anesthesia and then cutting of vena cava and removal of the heart.

### BAL Sampling

Broncheo-alveolar lavage (BAL) was performed by manual perfusion of the whole lung. After the trachea is exposed, a polyethylene tube (PE120) was inserted and ligated with 1-0 silk suture. The tube was connected to a syringe, prefilled with 4 ml of PBS at room temperature, and PBS was slowly injected into the lung. The fluid was be recollected by slow aspiration into the syringe. This procedure was be performed twice. Final BAL fluid will be transferred to a test tube (4 ml, polypropylene [PP]).

Tubes with BAL samples were weighed assuming that 1 gram is equal to 1 ml. BAL was ice chilled until centrifugation (Hettich ROTANTA 46R, 1200 rpm, 10 min, 4° C.). After centrifugation the supernatant was collected and divided onto 96-well plates (0.15 mL/well, 5 plates) and kept on dry ice (0.1 ml/well). Plates were stored at minimum of −75° C. for any further analysis. The cell pellet was be resuspended in 0.5 ml of PBS, kept on ice and immediately processed to the cell counting. The total and differential number of cells will be counted using automated SYSMEX XT-1800i Vet (Sysmex, Kobe Japan).

### Organ Collection

The right lung lobes were tied off and dissected out, trimmed off from non-pulmonary tissue, free from blood clots and rinsed with saline. The right lobes were abscised using a suture and cut loose and rinsed with PBS to remove any blood contamination. The superior lobe and middle lobe were weighed and collected in 7 mL precellys tubes for eGFP mRNA analysis. The inferior and post-caval lobe were weighed and collected in 7 mL precellys tubes for eGFP protein analysis). All right lobe samples are snap frozen in liquid nitrogen. Samples were saved and stored at minimum −80° C. until further processing for analysis. The left lobe was inflated with formalin and put into excess formalin in a container with plastic lid.

### Example 8. In Vivo Intravenous Administration to Mice of LNP-eGFP mRNA Formulations

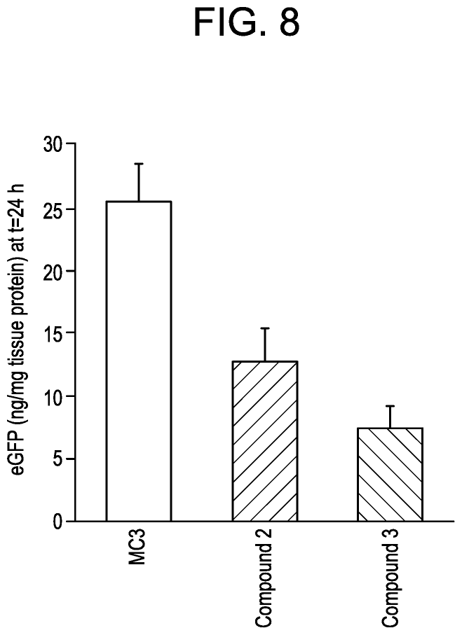

In compliance with the EU Directive 2010/63/EU, all work was carried out to Home Office U.K. ethical and husbandry standards and under the authority of a UK Project license, reviewed and approved by the Animal Welfare and Ethical Review Body (AWERB). Wild-type female BALB/c mice (6-8 weeks of age) were purchased from Charles River, UK and housed at the AstraZeneca animal for facility. All mice were injected with 100 μl of MC3, Compound 2 and Compound 3 LNP formulations were prepared by similar procedures described in Example 6, and the characterizations are shown in Table 2. The LNP formulations containing 0.4 mg/kg eGFP mRNA via intravenous tail vein injection. Mice were euthanised 24 hours after administration and the following organs were extracted: liver, spleen, lungs, kidneys and heart. Organs were collected into cryovials and snap frozen. The expression levels of eGFP in mouse organs at 24 hours are shown in FIGS. 8 (liver), 9 (spleen), 10 (lung), 11 (kidney) and 12 (heart).

TABLE 2
Characterization of LNP Formulations

| | | | Polymer- | | | |
|---|---|---|---|---|---|---|
| | Ionizable | N/P | conjugated | % | <d>_{N} | <d>_{Z} |
| Formulation # | lipid | ratio | lipid | EE | (nm) | (nm) |
| 6 | Compound 2 | 6 | DMPE-PEG2000 | 98 | 62 | 91 |
| 7 | Compound 3 | 6 | DMPE-PEG2000 | 97 | 120 | 143 |
| 8 | MC3 | 6 | DMPE-PEG2000 | 99 | 45 | 65 |

### Tissue Homogenisation

Organs/tissues homogenisation and cell lysis was carried out prior to ELISA. Briefly, organs/tissues were thawed on ice and rinsed with 1×DPBS to remove any blood. Organs/tissues were cut into approximately 100-200 mg slices and transferred to a 2 mL tube containing 0.5-1 mL of ice-cold 1× Cell Extraction Buffer PTR (from ELISA kit ab171581) with protease and phosphatase inhibitors (ThermoFisher 78445) and a 5 mm stainless steel bead (Qiagen). Organs/tissues samples were homogenised for 3 min on a Tissue Lyser II (Qiagen) set at a frequency of 30 1/s and then transferred to clean tubes and incubated on ice for 20 min. Samples were then centrifuged 20 min at 18000×g, 4° C. and clarified homogenates were transferred to a clean tube, aliquoted and stored at −80° C. until further use.

Quantification of eGFP in Organs by Ex Vivo ELISA

eGFP expression levels in the liver, spleen, lungs, kidneys and heart were measured using the GFP SimpleStep ELISA® Kit (ab171581) according to the manufacturer's instructions. All reagents were equilibrated to room temperature prior to use. The following buffers were prepared: 1× cell extraction buffer PTR with protease and phosphatase inhibitors (ThermoFisher 78445), 1× Wash buffer PT, 1× antibody diluent, antibody cocktail and EGFP standard curve. Where further dilutions of the tissue homogenates were needed, these were carried out in ice-cold 1× Cell extraction buffer PTR. Then, 50 μl of diluted samples and eGFP standards were added to respective wells, followed by the addition of 50 μl of the Antibody cocktail. The plate was then sealed and incubated for 1 h at room temperature on a plate shaker set to 400 rpm. Following incubation, each well was washed 3 times with 350 μl of 1× Wash buffer PT. After the final washing step, the excess liquid was removed by blotting the plate against clean paper towel. 100 μl of TMB substrate were then added to each well and plate was covered with aluminium foil and incubated for 10 min on a plate shaker set to 400 rpm. Finally, 100 μl of stop solution were added to each well, the plate was shaken for 1 min, and absorbance at 450 nm was recorded using an Envision microplate reader (Perkin Elmer). eGFP standard curve was fitted to a sigmoidal 4PL curve using GraphPad Prism 9 and ng eGFP protein were extrapolated from the curve. eGFP amount was normalised by total tissue protein, as determined by BCA assay (Pierce 23225) following manufacturer's instructions. Briefly, 25 μl of diluted tissue homogenates or BSA standards were added to respective wells, followed by the addition of 200 μl of working reagent to each well and contents were quickly mixed for 30 s on a plate shaker. The plate was then covered and incubated at 37° C. for 30 min. After cooling the plate to room temperature, the absorbance at 562 nm was recorded using an Envision microplate reader (Perkin Elmer). Total protein (mg) was extrapolated from the BSA standard curve. Data are reported as average ng eGFP/mg of tissue protein ±SD.

### Example 9. In Vivo Intravenous Administration to Mice of LNP Co-formulations of hEPO and Luciferase mRNA

Preparation of Lipid Nanoparticle (LNP) Formulations Containing hEPO and Luciferase mRNA:

A solution of hEPO (human EPO) mRNA and Luciferase mRNA (both purchased from TriLink Biotechnologies) was mixed in a 1:1 ratio in citrate buffer by mixing mRNA dissolved in nuclease free water, 100 mM citrate buffer (pH 3) and nuclease free water to give a solution of 50 mM citrate. Lipid solutions in ethanol (99.5%) was prepared by similar procedures described in Example 6 with four different lipid components: ionizable lipid, i.e., MC3 or Compound 2 or Compound 3; cholesterol (Sigma-Aldrich); DSPC (distearoyl phosphatidyl choline, Avanti Polar Lipids Inc); and DMPE-PEG2000 (dimyristoyl phosphatidyl ethanolamine-poly(ethylene glycol) 2000, NOF Corporation). Characterizations of the LNPs are shown in Table 3. The total concentration of lipids in all experiments was 12.5 mM. The mRNA and lipid solutions were mixed in a NanoAssemblr (Precision Nanosystems, Vancouver, BC, Canada) microfluidic mixing system at a mixing ratio of Aq:EtOH=3:1 and a constant flow rate of 12 mL/min. At the time of mixing the molar ratio of the ionizable lipid and phosphorus atoms on the mRNA chain was equal to 6. The first 0.2-0.35 mL and the last 0.05-0.1 mL of the LNP suspension prepared were discarded while the rest of the volume was collected as the sample fraction. The sample volume was transferred immediately to a Slide-a-lyzer G2 dialysis cassette (10000 MWCO, ThermoFischer Scientific Inc.) and dialyzed over night at 4° C. against PBS (pH7.4). The volume of the PBS buffer was 500-1000× the sample fraction volume. The next day, sample was taken from the cassette with a syringe and needle. The needle was then replaced by a 0.2 um syringe filter and the sample was filter sterilized in a sterile tube. From this sample 10 μl was diluted with 990 μl PBS buffer pH 7.4 and used to measure the intensity averaged particle size on a Malvern ZetaSizer (ZetaSizer Nano ZS, Malvern Instruments Inc., Westborough, MA, USA) and polydispersity index (PDI). The final mRNA concentration and encapsulation efficiency percentage (% EE) was measured by Quant-it Ribogreen Assay Kit (ThermoFischer Scientific Inc.) using Triton-X100 to disrupt the LNPs.

TABLE 3
Characterization of LNP Formulations

| | | | Polymer- | | | |
|---|---|---|---|---|---|---|
| | Ionizable | N/P | conjugated | % | <d>_{N} | <d>_{Z} |
| Formulation # | lipid | ratio | lipid | EE | (nm) | (nm) |
| 9 | Compound 2 | 6 | DMPE-PEG2000 | 98 | 46 | 63 |
| 10 | Compound 3 | 6 | DMPE-PEG2000 | 97 | 48 | 72 |
| 11 | MC3 | 6 | DMPE-PEG2000 | 98 | 49 | 65 |

### Animal Experiment:

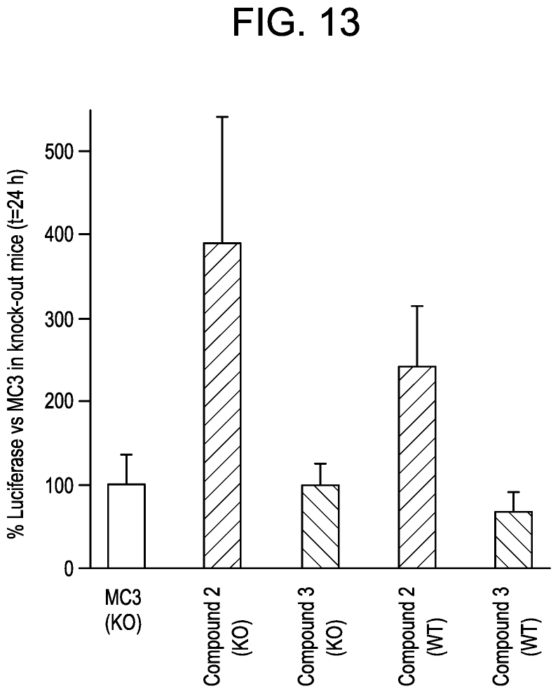

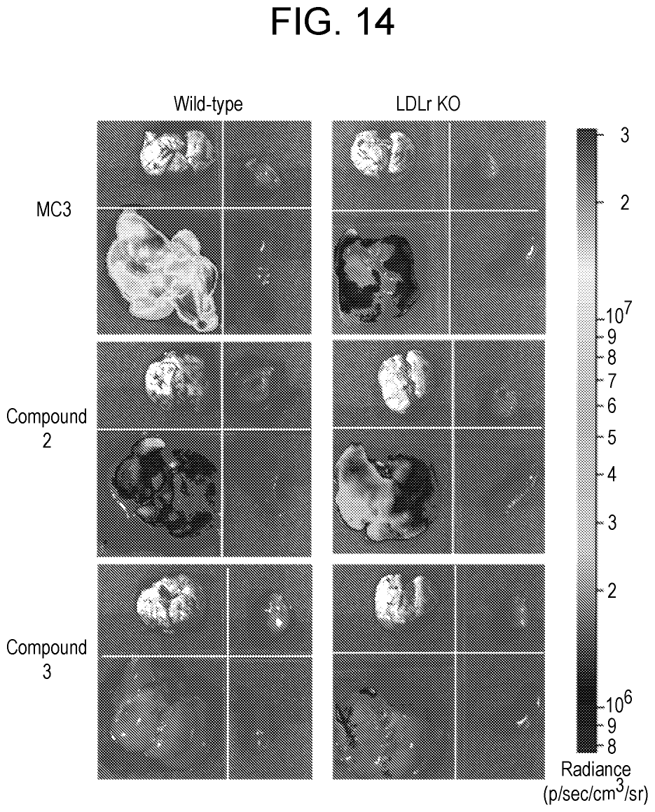

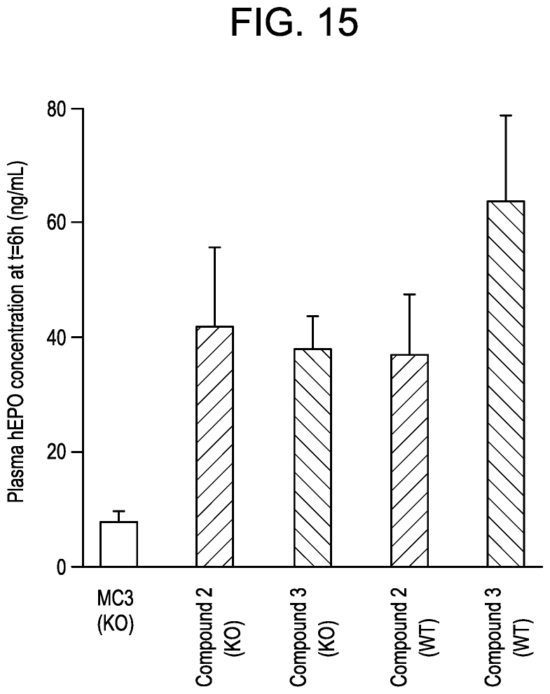

Experiments were performed in wild type (WT) and LDLr knockout (KO) mice. All experiments were performed in accordance with Swedish Animal Welfare and were approved by the Ethical Committee for Laboratory Animals in Gothenburg, Sweden. An equal number of male and female C57bl6 LDLR −/− mice and their wildtype littermates were bred in-house. Twenty C57bl6 LDLR −/− and 40 WT mice were included in the study (N≥5 for each group). Animals were used at an average bodyweight of 25 g. Animals were restrained and injected intravenously with 0.15 mg/kg hEPO mRNA+0.15 mg/kg Luc mRNA co-formulated in LNPs described above (0.3 mg/kg total mRNA) or with PBS as control. Blood samples were collected in EDTA tubes at t=6 h via tail vein bleeding and at t=24 h via orbital plexus bleeding before sacrifice. Mice were injected subcutaneously with 5 mg/kg luciferin substrate (RediJect D-Luciferin Bioluminescent Substrate from PerkinElmer) 20 minutes before termination. Heart, lung, spleen and liver were collected immediately after termination and imaged using an IVIS Spectrum (PerkinElmer). Total luminescence from each organ was quantified using LivingImage (PerkinElmer). The resulting IVIS images are shown in FIG. 14. For hEPO analysis, blood samples were spun down and Human Erythropoietin Quantikine IVD ELISA Kit (R&D System) was used to analyze plasma. Data are reported as hEPO in ng/ml and samples were analyzed in triplicates. FIG. 13 shows the expression of luciferase protein in WT and KO mice liver at 24 hours after intravenous administration of LNP formulations comprising MC3, Compound 2 and Compound 3, while FIG. 15 shows the hEPO protein concentration in WT and KO mice plasma at 6 hours after intravenous administration of LNP formulations comprising MC3, Compound 2 and Compound 3.

### Example 10. In Vivo Intrastriatal Administration to Mice of LNP Formulations of Luciferase mRNA

The MOD5 and Compound 5 (AZ8608) LNP formulations containing luciferase mRNA were prepared by the similar procedures as described in Examples 6 and 9 wherein the lipid components are MOD5/cholesterol/DSPC/DMPE-PEG=50/38.5/10/1.5 mol %; and Compound 5/cholesterol/DSPC/DMPE-PEG=42.5/40/16/1.5 mol %. Characterizations of the LNPs are shown in Table 4.

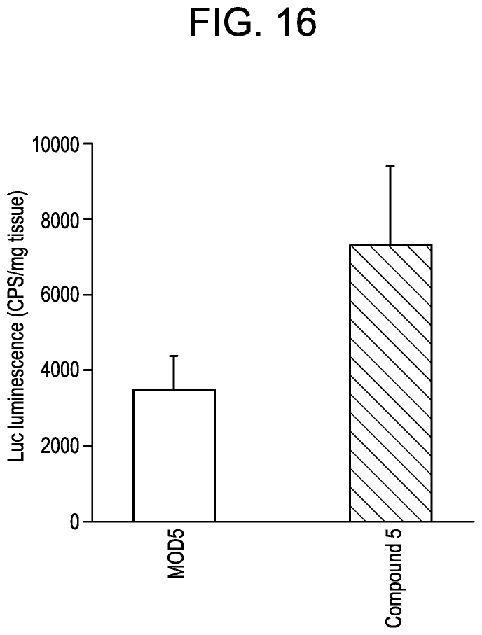

In-house generated reporter mice (LoxP Luc) containing a Cyclic recombinase enzyme (Cre) inducible luciferase expression cassette were generated by standard random integration gene addition transgenesis. Prior to LNP administration, the LoxP Luc reporter mice were anaesthetized with Isoflurane 4.0/1.5 O2 i. Mice were injected with 2 units (20 μl) diluted Comforion Vet (10 mg/ml; 100 μg/mouse) subcutaneous under the neck skin with an insulin syringe (BD U-100). The fur was next shaved and the skull washed with a Descutan swab (4% chlorhexidine) before placing the animals on the stereotactic board with a heating pad. The mice were properly attached, the skull was adjusted to a horizontal position, and covered with a plastic film with an open hole over the skull. During the surgery, a 6-8 mm incision was performed in the midline of the skull, drying the area around the bregma before placing the drill on that position after calculating the correct coordinates. One or two small holes were drilled through the skull bone on each side of the midline and a pump was attached with a Hamilton syringe to the stereotax. The syringe was placed over the drill hole, slowly lowered to the right depth and 5 μl of LNP solution were injected per hole at 0.5 μl/min flow injection pump rate. Mice were dosed 1.7 mg RNA (Cas9 mRNA/gRNA1/gRNA2 50/25/25 w/w) with 2 injections a 5 mL per mice into local brain striatum (one per hemisphere). The injector was kept in place for 3 minutes after each injection before slowly elevating the syringe and removing both pump and syringe. To close the skin above the drilled holes 2-3 stitches (Suture Polysorb 6-0) were used together with tissue glue which was placed along the incision. Animals were observed and weighed daily. Their recovery process and wound healing was closely followed for 3-5 days. After 7 days, ex vivo brain luciferase analysis of both hemispheres were performed. FIG. 16 illustrates the cortex and striatum average Luc expression level in LoxP Luc reporter mouse after intrastriatal administration of LNP formulations comprising MOD5 and Compound 5.

TABLE 4
Characterization of LNP Formulations

| | | | Polymer- | | | |
|---|---|---|---|---|---|---|
| | Ionizable | N/P | conjugated | % | <d>_{N} | <d>_{Z} |
| Formulation # | lipid | ratio | lipid | EE | (nm) | (nm) |
| 12 | Compound 5 | 2 | DMPE-PEG2000 | 96 | 51 | 70 |
| 13 | MOD5 | 3 | DMPE-PEG2000 | 86 | 57 | 74 |

### Protein Extraction and Luciferase Assay

One week post-LNP administration, the LoxP Luc reporter mice were sacrificed, the whole brain was dissected and striatum and cortex were isolated, weighed, placed in separate tubes and frozen until further analysis. To measure the LNP-mediated functional delivery levels, protein was extracted from the mouse brain tissues using a Qiagen TissueLyser as per manufacturer recommendation. The tissues were crushed and homogenized with a pestle driven and centrifuged to remove non-soluble tissue debris from the suspensions. The supernatants were transferred into separate tubes and protein concentration was determined by Bradford assay experiments. For the luciferase assays, 20 μl of each supernatant was added to a microplate (white OptiPlate-96) and 100 μl of D-luciferin were added to each well. Samples were mixed and the resulting luminescence signal was measured in a luminometer. The luminescence signal was normalized to tissue weight.

1. A compound of Formula (I): 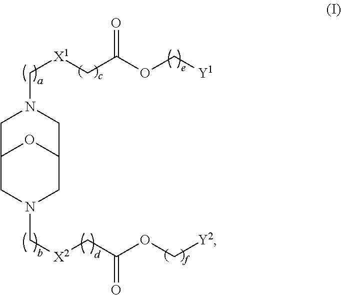
or a pharmaceutically acceptable salt thereof, wherein
a and b are each independently 3, 4 or 5;
c and d are each independently 1, 2 or 3;
e and f are each independently 0, 1 or 2;
X¹ is methylene or 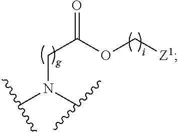
X² is methylene or 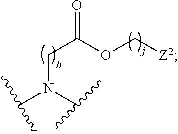
g and h are each independently 1, 2 or 3;
i and j are each independently 0, 1 or 2;
Y¹ and Y² are each independently straight-chain C₇₋₁₀ alkyl, straight-chain C₇₋₁₀ alkenyl, or 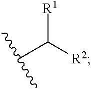
Z¹ and Z² are each independently straight-chain C₇₋₁₀ alkyl, straight-chain C₇₋₁₀ alkenyl, or 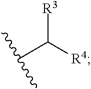
R¹ and R² are each independently straight-chain C₇₋₁₀ alkyl; and
R³ and R⁴ are each independently straight-chain C₇₋₁₀ alkyl.

2. The compound of claim 1, wherein X¹ and X² are both methylene.

3. The compound of claim 1 or 2, wherein a and b are both 4; and c and d are both 2.

4. The compound of any one of claims 1 to 3, wherein e and f are both 0.

5. The compound of any one of claims 1 to 4, wherein
Y¹ is straight-chain C₇₋₁₀ alkyl or straight-chain C₇₋₁₀ alkenyl;
Y² is 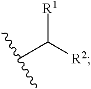
and
R¹ and R² are each independently straight-chain C₇₋₁₀ alkyl.

6. The compound of any one of claims 1 to 4, wherein Y¹ and Y² are each independently 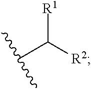
and
R¹ and R² are each independently straight-chain C₇₋₁₀ alkyl.

7. The compound of any one of claims 1 to 3, wherein e and f are each independently 1 or 2.

8. The compound of claim 7, wherein Y¹ and Y² are each independently 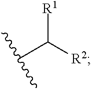
and
R¹ and R² are each independently straight-chain C₇₋₁₀ alkyl.

9. The compound of any one of claims 1 to 6 and 8, which is of Formula (II): 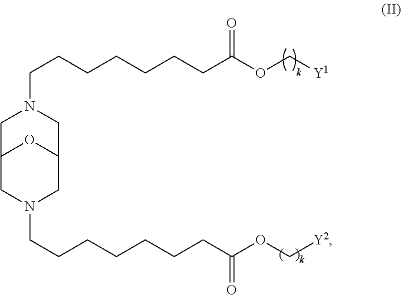
wherein
k is 0, 1 or 2;
Y¹ is straight-chain C₇₋₁₀ alkyl, straight-chain C₇₋₁₀ alkenyl, or 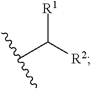
Y² is 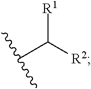
R¹ and R² are each independently straight-chain C₇₋₁₀ alkyl.

10. The compound of claim 1, wherein
X¹ is 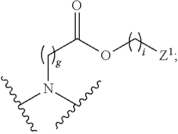
and
X² is

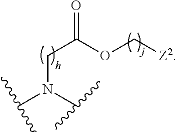

11. The compound of claim 10, wherein a and b are both 4; and c and d are both 2.

12. The compound of claim 10 or 11, wherein e and f are each independently 1 or 2.

13. The compound of any one of claims 10 to 12, wherein g and h are both 2.

14. The compound of any one of claims 10 to 13, wherein i and j are both 1.

15. The compound of any one of claims 10 to 14, wherein Y¹ and Y² are each independently 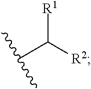
and
R¹ and R² are each independently straight-chain C₇₋₁₀ alkyl.

16. The compound of claim 1, which is selected from:
heptadecan-9-yl 8-(7-(8-(nonyloxy)-8-oxooctyl)-9-oxa-3,7-diazabicyclo[3.3.1]nonan-3-yl)octanoate;
heptadecan-9-yl (Z)-8-(7-(8-(non-2-en-1-yloxy)-8-oxooctyl)-9-oxa-3,7-diazabicyclo[3.3.1]nonan-3-yl)octanoate;
3-heptyldodecyl 8-(7-(8-((3-octylundecyl)oxy)-8-oxooctyl)-9-oxa-3,7-diazabicyclo[3.3.1]nonan-3-yl)octanoate;
di(heptadecan-9-yl) 8,8′-(9-oxa-3,7-diazabicyclo[3.3.1]nonane-3,7-diyl)dioctanoate; and
tetrakis(2-octyldecyl) 3,3′,3″,3″′-(((9-oxa-3,7-diazabicyclo[3.3.1]nonane-3,7-diyl)bis(butane-4,1-diyl))bis(azanetriyl))tetrapropionate;
or a pharmaceutically acceptable salt thereof.

17. A lipid nanoparticle comprising the compound of any one of claims 1 to 16, or a pharmaceutically acceptable salt thereof.

18. The lipid nanoparticle of claim 17, further comprising at least one neutral lipid, at least one sterol and at least one polymer-conjugated lipid.

19. The lipid nanoparticle of claim 18, wherein the neutral lipid is selected from distearoyl phosphatidylcholine (DSPC), dioleoyl phosphatidylethanolamine (DOPE), dipalmitoyl phosphatidylcholine (DPPC), dimyristoyl phosphatidylcholine (DMPC) or combinations thereof.

20. The lipid nanoparticle of claim 18 or 19, wherein the sterol is cholesterol.

21. The lipid nanoparticle of any one of claims 18, 19 and 20, wherein the polymer-conjugated lipid is selected from DMPE-PEG2000, DPPE-PEG2000, DMG-PEG2000, DPG-PEG2000, PEG2000-c-DOMG, PEG-C-DOPG or combinations thereof.

22. The lipid nanoparticle of any one of claims 17 to 21, wherein the lipid nanoparticle further comprises distearoyl phosphatidylcholine (DSPC), cholesterol, and DMPE-PEG2000.

23. The lipid nanoparticle of any one of claims 17 to 22, further comprising a therapeutic agent.

24. The lipid nanoparticle of claim 23, wherein the therapeutic agent is a nucleic acid segment.

25. The lipid nanoparticle of claim 24, wherein the nucleic acid segment is an RNA.

26. The lipid nanoparticle of claim 25, wherein the RNA is a modified mRNA.

27. A pharmaceutical composition comprising a plurality of the lipid nanoparticles of any one of claims 23 to 26.

28. A method of treating a disease or disorder in a subject comprising administering to the subject an effective amount of the pharmaceutical composition of claim 27.

29. Use of the pharmaceutical composition of claim 28 to treat a disease or disorder.

30. The pharmaceutical composition of claim 27 for use in the treatment of a disease or disorder.
